(19) United States
(12) Patent Application Publication
(10) Pub. No.: US 2018/0014742 A1
(43) Pub. Date: Jan. 18, 2018

(54) BIOLOGICAL INFORMATION MEASURING DEVICE

(71) Applicant: SEIKO EPSON CORPORATION, Tokyo (JP)

(72) Inventor: Takanori IWAWAKI, Hachioji-shi (JP)

(73) Assignee: SEIKO EPSON CORPORATION, Tokyo (JP)

(21) Appl. No.: 15/547,870

(22) PCT Filed: Jan. 25, 2016
(86) PCT No.: PCT/JP2016/000358

§ 371 Date: Aug. 1, 2017

(30) Foreign Application Priority Data
Feb. 25, 2015 (JP) 2015-035755

(51) Int. Cl. A61B 5/0408; A61B 5/0428; A61B 5/0245; A61B 5/044; A61B 5/00

(52) U.S. Cl. CPC A61B 5/04085; A61B 5/044; A61B 5/681; A61B 5/0245; A61B 5/0428; A61B 2562/0209

(57) ABSTRACT

A biological information measuring device includes a biological information detection unit, a control unit, and a casing. The biological information detection unit includes a first surface-side electrode having electrodes which can come into contact with the user, a second surface-side electrode having electrodes which are exposed on a second surface and come into contact with the user, and an electrocardiographic detection unit. At least one of the first surface-side electrode and the second surface-side electrode has a plurality of electrodes. The control unit sets one electrode of the first surface-side electrode and one electrode of the second surface-side electrode as working electrodes, sets, as a reference electrode, one of the electrodes that are not set as the working electrodes, and causes the electrocardiographic detection unit to measure an electrocardiogram of the user, based on a current detected with the working electrodes.

### TECHNICAL FIELD

The present invention relates to a biological information measuring device.

### BACKGROUND ART

Traditionally, a wristwatch-type electronic blood pressure monitor as a biological information detection device which is configured to be portable and detests biological information of the user is known (see, for example, PTL 1).

This wristwatch-type electronic blood pressure monitor disclosed in PTL 1 is to be worn around the user's wrist or the like with a strap, and exchanges an optical signal with a transfer device connected to a blood pressure measuring device and acquires blood pressure data of systolic blood pressure and diastolic blood pressure measured by the blood pressure measuring device. This wristwatch-type electronic blood pressure monitor includes a main body case made of an electrically insulating synthetic resin, and a display unit, an optical element unit, an electrocardiographic wave detection electrode, a back cover and a circuit board which are provided in the main body case.

Of these, the display unit displays the systolic blood pressure and diastolic blood pressure that are acquired.

The optical element unit has an LED (light emitting diode) and a phototransistor, and detects the pulse of the user from the user's finger applied thereto.

The electrocardiographic wave detection electrode and the back cover are electrodes for detecting electrocardiographic waves. The electrocardiographic wave detection electrode is provided on the front side of the main body case. The back cover is provided on the back side of the main body case, that is, at a position that comes in contact with the user when the main body case is worn by the user.

The circuit board is connected to the display unit, the optical element unit, the electrocardiographic detection electrode and the back cover, and controls the operations of these.

With this wristwatch-type electronic blood pressure monitor, the subject can easily measure an electrocardiogram by him/herself, compared with a device which monitors and records an electrocardiogram detected with electrodes attached to the four limbs and chest of the subject.

### CITATION LIST

### Patent Literature

PTL 1: JP-A-7-88090

### SUMMARY OF INVENTION

### Technical Problem

However, in the wristwatch-type electronic blood pressure monitor disclosed in the foregoing PTL 1, the electrocardiographic wave detection electrode and the back cover are determined in advance as the electrodes used to measure an electrocardiogram. Therefore, if grounding is not appropriate, irregularities, for example, baseline drift (fluctuations of waveform), may occur in electrocardiographic waveforms and there is a risk of not being able to detect an electrocardiogram properly. Meanwhile, the easiness of setting the grounding depends on the arrangement of electrodes. If the arrangement of the electrodes is not appropriate, there is a risk that user-friendliness may be lowered.

Therefore, a configuration which enables improvement in the detection accuracy of electrocardiograms has been demanded.

An object of the invention is to solve at least a part of the foregoing problems and to provide a biological information measuring device which enables improvement in the detection accuracy of biological information.

### Solution to Problem

A biological information measuring device according to an aspect of the invention includes: a biological information detection unit which detects biological information of a user; a control unit which controls the biological information detection unit; and a casing which houses the biological information detection unit and the control unit. The biological information detection unit includes a first surface-side electrode arranged on a first surface of the casing, a second surface-side electrode arranged on a second surface which is a different surface from the first surface of the casing, and an electrocardiographic detection unit which detects an electrocardiogram of the user, using the first surface-side electrode and the second surface-side electrode. At least one of the first surface-side electrode and the second surface-side electrode has a plurality of electrodes. The control unit sets one electrode of the first surface-side electrode and one electrode of the second surface-side electrode as working electrodes, sets, as a reference electrode, one of the electrodes that are not set as the working electrodes, and causes the electrocardiographic detection unit to measure an electrocardiogram of the user, based on a current detected with the working electrodes.

The reference electrode refers to a grounding electrode.

With the above configuration, one of the electrodes provided in the first surface-side electrode and one of the electrodes provided in the second surface-side electrode are set as working electrodes, and one of the electrodes that are not set as the working electrodes is set as a reference electrode. Thus, not only working electrodes suitable for measuring an electrocardiogram can be set but also an appropriate electrode can be set as a reference electrode. Therefore, since the electrocardiographic detection unit detects an electrocardiogram of the user, using these working electrodes and reference electrode, the detection accuracy of the electrocardiogram can be improved and the electrocardiogram can be detected and measured with high accuracy.

In the above configuration, it is preferable that the control unit sets the reference electrode, based on an impedance value based on a voltage value of a current which is outputted to the first surface-side electrode and electrically continues to the second surface-side electrode.

The impedance value is an impedance value in a path through which a current electrically flows between an electrode of the first surface-side electrode and an electrode of the second surface-side electrode via the human body of the user, that is, a bioelectrical impedance value.

According to the configuration, since a reference electrode can be set based on the impedance value that is actually detected in the path electrically continuing between an electrode of the first surface-side electrode and an electrode of the second surface-side electrode, an appropriate reference electrode can be set. Therefore, the detection accuracy of the electrocardiogram can be improved further and the electrocardiogram can be detected and measured more accurately.

In the above configuration, it is preferable that the control unit sets the working electrodes and the reference electrode used for measurement of an electrocardiogram of the user, based on an electrocardiogram of the user detected with at least one combination of electrodes in which one electrode of the first surface-side electrode and one electrode of the second surface-side electrode are set as provisional working electrodes and in which one of the electrodes that are not set as the provisional working electrodes is set as a provisional reference electrode.

As combinations of provisional working electrodes and a provisional reference electrode, for example, eight combinations may be considered if the first surface-side electrode and the second surface-side electrode have two electrodes each.

Here, irregularities occur in electrocardiographic waveforms as described above, not only in the case where a proper reference electrode is not selected but also in the case where, for example, there is an influence of an electromagnetic induction noise or the like.

In contrast, according to the above configuration, the working electrodes and the reference electrode are set, based on an electrocardiogram (for example, electrocardiographic waveforms) detected with at least one combination of the provisional working electrodes and the provisional reference electrode. Thus, proper working electrodes and reference electrode can be selected and set, based on an electrocardiogram that is actually detected. Therefore, the detection accuracy of the electrocardiogram can be improved further and the electrocardiogram can be detected and measured more accurately.

In the above configuration, it is preferable that the casing includes a main body part and a wearing member which allows the main body part to be worn at a wearing site of the user, that the second surface is a surface in contact with the wearing site when the main body part is worn at the wearing site with the wearing member, and that the first surface is a surface opposite to the second surface.

As an example of the wearing member, a strap that can be wound on a human body can be employed.

With the above configuration, since the second surface is a surface on the wearing site side of the main body part, the second surface-side electrode arranged on the second surface can be securely brought into contact with the human body of the user. Also, since the first surface is a surface opposite to the second surface, it can be made easier to bring the first surface-side electrode arranged on the first surface into contact with the human body of the user by placing a hand thereon or the like. Therefore, the detection and measurement of the electrocardiogram of the user can be carried out in a simple way, and the electrically continuous path between the first surface-side electrode and the second surface-side electrode can be made longer, and therefore, the detection accuracy of the electrocardiogram can be improved.

In the above configuration, it is preferable that a display unit arranged on the first surface of the main body part is provided, that the main body part has an electrode arrangement part which is arranged on the first surface and surrounds the display unit, and that the first surface-side electrode is arranged in the electrode arrangement part.

With the above configuration, since the first surface-side electrode is arranged in the electrode arrangement part surrounding the display unit arranged on the first surface, the first surface-side electrode can be arranged without limiting the arrangement of the display unit even in the case where the first surface-side electrode is made up of a plurality of electrodes. Also, in such a case, since the plurality of electrodes forming the first surface-side electrode can be arranged separately from each other along the electrode arrangement part, it can be made easier to make the respective electrodes electrically independent of each other. Moreover, with the display unit, the detected biological information such as an electrocardiogram can be presented to the user.

In the above configuration, it is preferable that the first surface-side electrode includes a plurality of electrodes, and that the plurality of electrodes is separated from each other at a position between a direction of 4 o'clock and a direction of 5 o'clock and a position between a direction of 10 o'clock and a direction of 11 o'clock, as viewed from a position directly opposite the first surface.

With the above configuration, the plurality of electrodes provided in the first surface-side electrode is arranged respectively on the lower left-hand side and the upper right-hand side of the first surface. Thus, for example, when the biological information measuring device is worn on the left wrist, it can be made easier to bring fingers (forefinger and middle finger) of the right hand into contact with the plurality of electrodes. Therefore, it can be made easier to carry out the detection and measurement of the electrocardiogram.

In the above configuration, it is preferable that the biological information detection unit has a pulse wave detection unit which detects a pulse wave of the user.

With the above configuration, the pulse wave of the user is detected as well as the electrocardiogram of the user. Therefore, since the biological information of the user that is detected can be increased, the versatility and convenience of the biological information measuring device can be improved.

### BRIEF DESCRIPTION OF DRAWINGS

FIG. 1 is a front view showing a biological information measuring device according to a first embodiment of the invention.

FIG. 2 is a back view showing the biological information measuring device in the first embodiment.

FIG. 3 is a block diagram showing the configuration of the biological information measuring device in the first embodiment.

FIG. 4 is a block diagram showing the configuration of a measuring unit in the first embodiment.

FIG. 5 is a block diagram showing the configuration of an electrocardiographic measuring unit and an impedance measuring unit in the first embodiment.

FIG. 6 is a block diagram showing the configuration of a control unit in the first embodiment.

FIG. 7 is a view showing an example of pulse wave and electrocardiographic waveform in the first embodiment.

FIG. 8 is a flowchart showing electrocardiographic measurement processing in the first embodiment.

FIG. 9 is a schematic view showing the wearing state of the biological information measuring device in the first embodiment.

FIG. 10 is a front view showing a biological information measuring device in a modification of the first embodiment.

FIG. 11 is a front view showing a biological information measuring device according to a second embodiment of the invention.

FIG. 12 is a back view showing a biological information measuring device according to a third embodiment of the invention.

FIG. 13 is a cross-sectional view showing a main body part and a light-transmitting member in the third embodiment.

FIG. 14 is a back view showing a biological information measuring device according to a fourth embodiment of the invention.

FIG. 15 is a cross-sectional view showing the biological information measuring device in the fourth embodiment.

### DESCRIPTION OF EMBODIMENTS

### First Embodiment

Hereinafter, a first embodiment of the invention will be described, based on the drawings.

### [Schematic Configuration of Biological Information Detection Device]

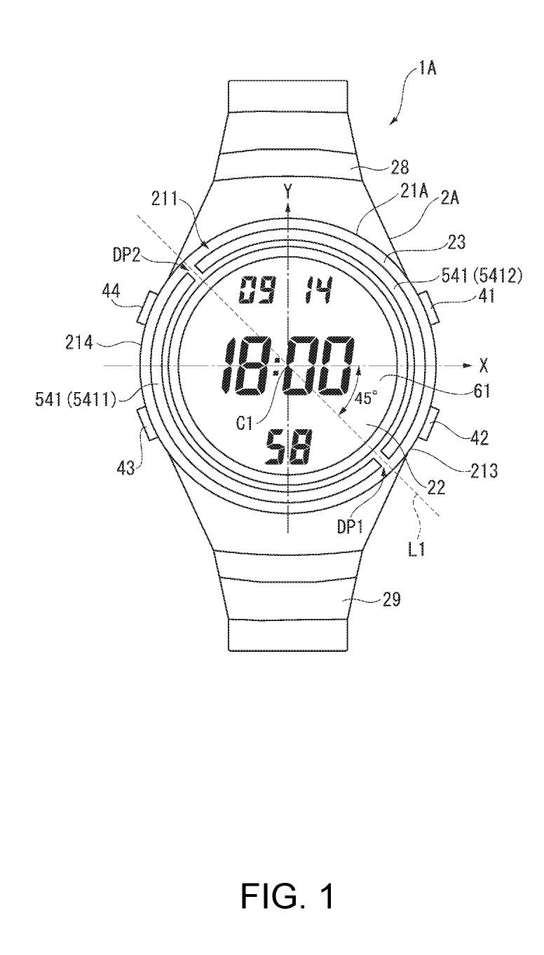

FIG. 1 is a front view showing a biological information measuring device 1A according to this embodiment.

The biological information measuring device (hereinafter abbreviated as the measuring device in some cases) 1A according to this embodiment is a wearable device used, worn at a wearing site such as a wrist of a user, and detects and stores biological information of the user. Specifically, the measuring device 1A detects a pulse wave and electrocardiogram as biological information of the user, stores the electrocardiogram, and also calculates a pulse rate based on the detected pulse wave and stores the pulse rate.

The measuring device 1A like this includes: a casing 2A having a main body part 21A and a pair of straps 28, 29; and a device main body 3 housed in the casing 2A, as shown in FIG. 1.

The pair of straps 28, 29 is equivalent to the wearing member of the invention. The pair of straps 28, 29 is connected to one end and the other end in the longitudinal direction of the main body part 21A and extends in the opposite directions to each other from the main body part 21A. This pair of straps 28, 29 is configured to be able to be fixed by a buckle (not illustrated) provided at a distal end of the strap 28 (end part on the side opposite to the connecting site to the main body part 21A). As the straps 28, 29 are thus fixed, the main body part 21A is worn at the wearing site. The straps 28, 29 may be integrated with the main body part 21A. In this case, the main body part 21A serves as the casing 2A.

The main body part 21A houses the device main body 3, described later. This main body part 21A has a back surface 212, which is a surface coming in contact with the body of the user when the measuring device 1A is worn on the body of the user, a front surface 211, which is a surface opposite the back surface 212, and a right lateral surface 213 and a left lateral surface 214 connecting these. That is, the back surface 212, in the main body part 21A, is a surface where a pulse wave sensor 531 of a pulse wave detection unit 53, described later, is arranged, or a surface where a light-transmitting member of the pulse wave sensor 531 is arranged. The front surface 211 is a surface on the side opposite to the back surface 212.

Of these, substantially at the center of the front surface 211 (equivalent to the first surface), a display unit 61 forming the device main body 3 is provided. The display unit 61 is covered by a circular cover 22. The front surface 211 is a surface on the side opposite to the back surface 212, as viewed along a normal line to the display surface of the display unit 61. Therefore, the front surface 211 may be a flat surface or may have a partly curved surface or recessed/protruding surface.

Also, a ring-shaped electrode arrangement part 23 surrounding the display unit 61 and the cover 22 is provided on the front surface 211. In this electrode arrangement part 23, a front surface-side electrode 541 forming an electrocardiographic measuring unit 54 of a measuring unit 5, described later, is arranged. The electrode arrangement part 23 also functions as a bezel.

The front surface-side electrode 541 is equivalent to the first surface-side electrode of the invention and has two electrodes 5411, 5412. These electrodes 5411, 5412 are each formed in the shape of a semicircular arc and are insulated from each other by an insulating material such as rubber in the electrode arrangement part 23. The arrangement of these electrodes 5411, 5412 will be described in detail later.

On the right lateral surface 213 and the left lateral surface 214, buttons 41 to 44 of an operation unit 4 forming the device main body 3 are arranged. These buttons 41 to 44 are buttons which project from and retract into the main body part 21A.

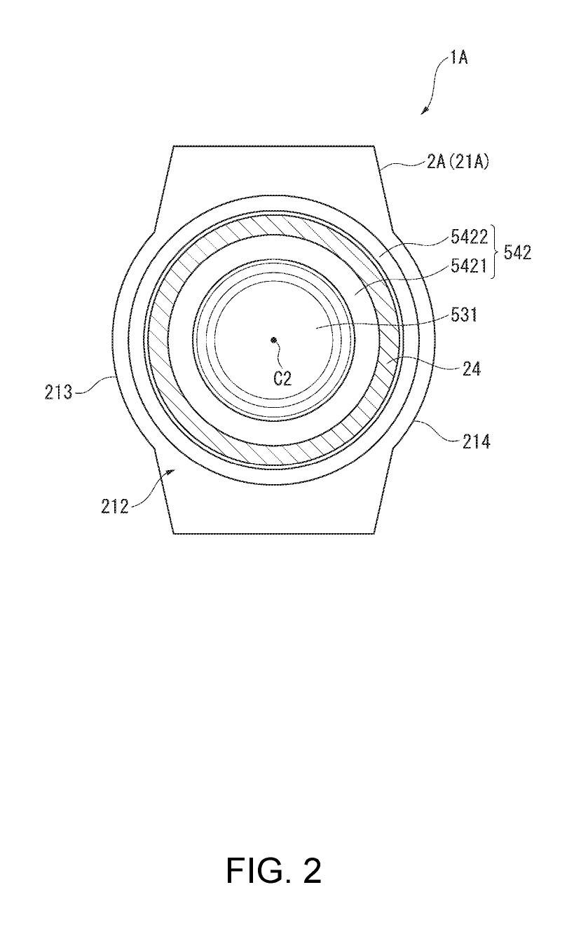

FIG. 2 is a back view showing the measuring device 1A, and more specifically, a view showing the back surface 212 of the main body part 21A.

The back surface 212 (equivalent to the second surface) is a surface facing the wearing site when the measuring device 1A is worn at the wearing site. On this back surface 212, the pulse wave sensor 531 and a back surface-side electrode 542 forming the electrocardiographic measuring unit 54 are exposed.

The pulse wave sensor 531 is a substantially circular sensor forming the pulse wave detection unit 53 of the measuring unit 5 and is arranged substantially at the center of the back surface 212. The pulse wave sensor 531 maybe arranged directly on the back surface 212. Also, the pulse wave sensor 531 may be provided in the device main body 3 provided inside the main body part 21A, and a light-transmitting member which covers a light emitting element and a light receiving element of the pulse wave sensor 531 may be attached to the back surface 212.

The back surface-side electrode 542 is equivalent to the second surface-side electrode of the invention and has two electrodes 5421, 5422. Of these, the electrode 5421 is formed in a substantially circular shape and is arranged, exposed at a position surrounding the pulse wave sensor 531. Meanwhile, the electrode 5422 is formed in a substantially circular shape and is arranged, exposed via an insulator 24 at a position surrounding the electrode 5421.

That is, the electrodes 5421, 5422 are respectively arranged concentrically about a center C2 of the circular pulse wave sensor 531.

### [Configuration of Device Main Body]

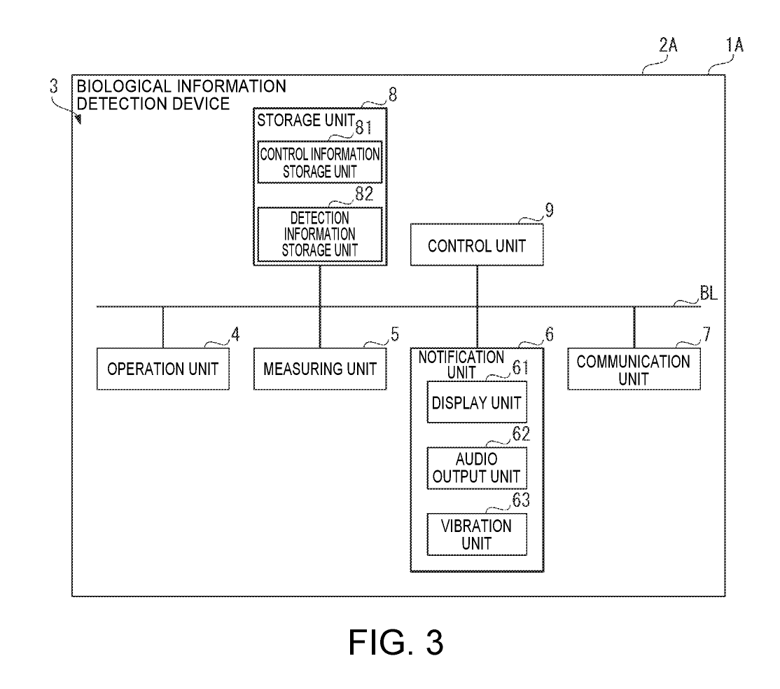

FIG. 3 is a block diagram showing the configuration of the measuring device 1A.

The device main body 3 is configured, having the operation unit 4, the measuring unit 5, a notification unit 6, a communication unit 7, a storage unit 8, and a control unit 9, as shown in FIG. 3.

### [Configuration of Operation Unit]

The operation unit 4 has the buttons 41 to 44 and outputs operation signals corresponding to input operations to these buttons 41 to 44, to the control unit 9. The operation unit 4 is not limited to a configuration with buttons and may also employ a configuration with a touch panel arranged on the display unit 61 of the notification unit 6, described later, or a configuration for detecting a tap operation by the user.

### [Configuration of Measuring Unit]

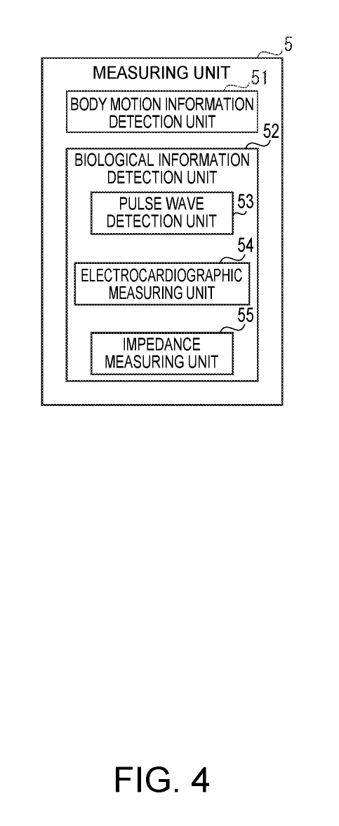

FIG. 4 is a block diagram showing the configuration of the measuring unit 5.

The measuring unit 5 has a body motion information detection unit 51 and a biological information detection unit 52, each of which operates under the control of the control unit 9.

The body motion information detection unit 51 detects body motion information indicating a body motion of the user and outputs the body motion information to the control unit 9. In this embodiment, the body motion information detection unit 51 detects an acceleration signal which changes with the body motion of the user, as body motion information. The body motion information detection unit 51 may detect an angular velocity which changes with the body motion of the user, in addition to the acceleration.

### [Configuration of Biological Information Detection Unit]

The biological information detection unit 52 detects biological information of the user. In this embodiment, the biological information detection unit 52 includes the pulse wave detection unit 53, the electrocardiographic measuring unit 54, and an impedance measuring unit 55.

### [Configuration of Pulse Wave Detection Unit]

The pulse wave detection unit 53 has the pulse wave sensor 531 and detects a pulse wave of the user under the control of the control unit 9. This pulse wave sensor 531 is a photoelectric sensor having a light emitting element such as an LED (light emitting diode), a light receiving element such as a photodiode, and a light-transmitting member which covers these, though not illustrated. In this pulse wave sensor, light cast from the light emitting element toward a living body is received by the light receiving element via blood vessels in the living body. A signal indicating change with time in the amount of light received by this light receiving element is outputted as a pulse wave signal to the control unit 9, described later, and the control unit 9 analyzes the pulse wave signal. Thus, a pulse rate is calculated.

### [Configuration of Electrocardiographic Measuring Unit]

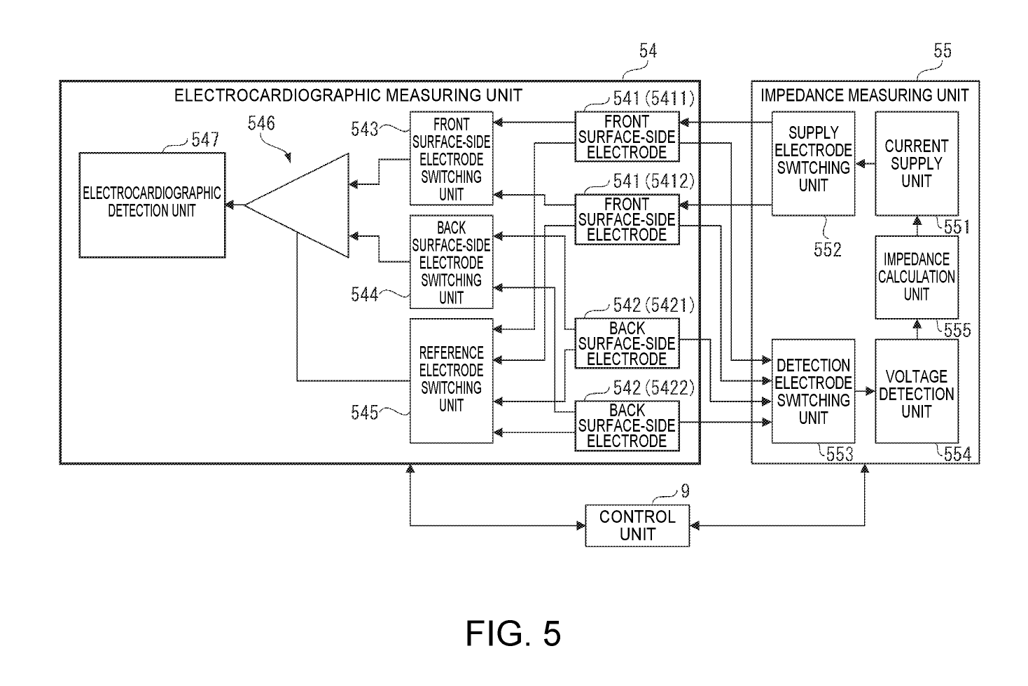

FIG. 5 is a block diagram showing the configuration of the electrocardiographic measuring unit 54 and the impedance measuring unit 55.

The electrocardiographic measuring unit 54 detects an electrocardiogram of the user and outputs an electrocardiographic signal indicating the electrocardiogram to the control unit 9. This electrocardiographic measuring unit 54 includes a front surface-side electrode switching unit 543, a back surface-side electrode switching unit 544, a reference electrode switching unit 545, an operational amplifier 546, and an electrocardiographic detection unit 547, in addition to the front surface-side electrode 541 and the back surface-side electrode 542, as shown in FIG. 5.

The front surface-side electrode switching unit 543 and the back surface-side electrode switching unit 544 switch the electrodes to be connected to two input terminals of the operational amplifier 546 under the control of the control unit 9.

Specifically, the front surface-side electrode switching unit 543 electrically connects one of the electrodes 5411, 5412 of the front surface-side electrode 541 connected to the switching unit 543, to one of the two input terminals of the operational amplifier 546.

Meanwhile, the back surface-side electrode switching unit 544 electrically connects one of the electrodes 5421, 5422 of the back surface-side electrode 542 connected to the switching unit 544, to the other of the two input terminals of the operational amplifier 546.

The reference electrode switching unit 545 switches the electrode to be connected to a grounding terminal of the operational amplifier 546, from among the electrodes 5411, 5412 of the front surface-side electrode 541 and the electrodes 5421, 5422 of the back surface-side electrode 542 connected to the switching unit 545, under the control of the control unit 9.

Such switching of electrodes by the front surface-side electrode switching unit 543, the back surface-side electrode switching unit 544 and the reference electrode switching unit 545 will be described in detail later.

The operational amplifier 546 has two input terminals (inverting input terminal and non-inverting input terminal), one grounding terminal, and one output terminal, as described above. As described above, one of the electrodes 5411, 5412 is connected to one input terminal of the operational amplifier 546 by the front surface-side electrode switching unit 543, and one of the electrodes 5421, 5422 is connected to the other input terminal by the back surface-side electrode switching unit 544. Also, an electrode that is not connected to the two input terminals, of the electrodes 5411, 5412, 5421, 5422, is connected to the grounding terminal of the operational amplifier 546 by the reference electrode switching unit 545.

This operational amplifier 546 amplifies signals inputted to the electrodes connected to the respective input terminals and outputs the amplified signals from the output terminal.

The electrocardiographic detection unit 547 is a signal processing unit which processes a signal inputted from the operational amplifier 546 and outputs an electrocardiographic signal based on that signal, to the control unit 9. Specifically, the electrocardiographic detection unit 547 filters the inputted signal to eliminate a noise component, and outputs the resulting electrocardiographic signal to the control unit 9.

### [Configuration of Impedance Measuring Unit]

The impedance measuring unit 55, using one of the electrodes 5411, 5412 of the front surface-side electrode 541 and one of the electrodes 5421, 5422 of the back surface-side electrode 542, measures an impedance value between these electrodes under the control of the control unit 9. This impedance measuring unit 55 has a current supply unit 551, a supply electrode switching unit 552, a detection electrode switching unit 553, a voltage detection unit 554, and an impedance calculation unit 555.

The current supply unit 551 is electrically connected to a power source, not illustrated, and to the supply electrode switching unit 552. The current supply unit 551 transforms the voltage of a current supplied from the power source and supplies the resulting current to the supply electrode switching unit 552.

The supply electrode switching unit 552 is connected to each of the electrodes 5411, 5412. Then, the supply electrode switching unit 552 supplies the current supplied from the current supply unit 551, to one of the electrodes 5411, 5412 under the control of the control unit 9.

The detection electrode switching unit 553 is connected to each of the electrodes 5411, 5412, 5421, 5422. Then, the detection electrode switching unit 553 switches the electrode to be electrically connected to the voltage detection unit 554, from among these electrodes 5411, 5412, 5421, 5422, under the control of the control unit 9.

The switching of electrodes by the supply electrode switching unit 552 and the detection electrode switching unit 553 will be described in detail later.

The voltage detection unit 554 detects the voltage value of the current inputted via the detection electrode switching unit 553, that is, of the current inputted from the electrode switched by the detection electrode switching unit 553. Then, the voltage detection unit 554 outputs the detected voltage value to the impedance calculation unit 555.

The impedance calculation unit 555 calculates an impedance value between the electrode selected by the supply electrode switching unit 552 and the electrode selected by the detection electrode switching unit 553 (that is, a bioelectrical impedance value), based on the voltage value of the current supplied by the current supply unit 551 and the voltage value of the current detected by the voltage detection unit 554. In this case, the impedance calculation unit 555 calculates the impedance value for each combination of an electrode switchable by the supply electrode switching unit 552 and an electrode switchable by the detection electrode switching unit 553. Then, the impedance calculation unit 555 outputs the calculated impedance value to the control unit 9.

### [Configuration of Notification Unit]

Back to FIG. 3, the notification unit 6 notifies the user of various kinds of information under the control of the control unit 9. This notification unit 6 has the display unit 61, an audio output unit 62, and a vibration unit 63.

The display unit 61 has various types of display panels such as liquid crystal and displays information inputted from the control unit 9. For example, the display unit 61 displays body motion information and biological information (pulse rate and electrocardiogram) detected and analyzed by the measuring unit 5. Moreover, the display unit 61 displays presentation information generated by the control unit 9.

The audio output unit 62 is configured with an audio output measure such as a speaker and outputs an audio corresponding to an audio signal inputted from the control unit 9.

The vibration unit 63 has a motor with its operation controlled by the control unit 9 and notifies the user of, for example, a warning through vibration generated by the driving of the motor.

### [Configuration of Communication Unit]

The communication unit 7 has a communication module capable of communicating with an external device. This communication unit 7 periodically transmits body motion information and biological information which are detected and measured, respectively, to the external device, and also outputs information received from the external device to the control unit 9. In this embodiment, the communication unit 7 wirelessly communicates with the external device by a short-range wireless communication system. However, the communication unit 7 may communicate with the external device via a relay device such as a cradle, and a cable. Moreover, the communication unit 7 may communicate with the external device via a network.

### [Configuration of Storage Unit]

The storage unit 8 is made up of a storage measure such as a flash memory and has a control information storage unit 81 and a detection information storage unit 82.

The control information storage unit 81 stores control information such as various programs and data which are necessary for the operations of the measuring device 1A. As such programs, a control program to control the measuring device 1A and an electrocardiographic measurement program to execute electrocardiographic measurement processing, described later, are stored.

The detection information storage unit 82 stores body motion information and biological information detected by the measuring unit 5, and the result of analysis of the body motion information and the biological information by the control unit (for example, pulse rate and electrocardiogram). This detection information storage unit 82 is configured to sequentially store these items of information and to overwrite the earliest stored information with the newly acquired information if the storage capacity has run short.

### [Configuration of Control Unit]

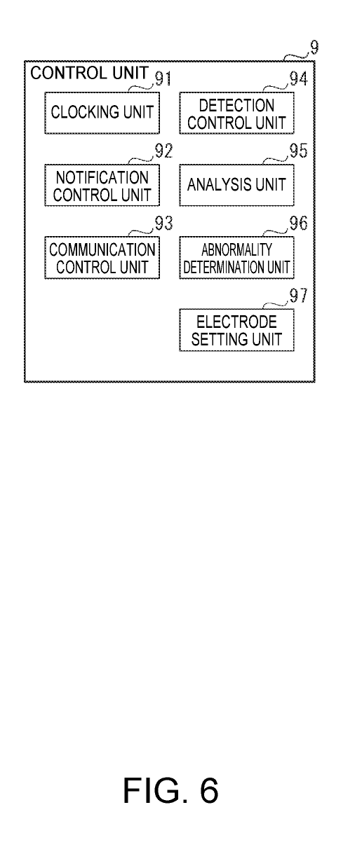

FIG. 6 is a block diagram showing the configuration of the control unit 9.

The control unit 9 has a processing circuit and controls the operations of the measuring device 1A autonomously or in response to an operation signal inputted from the operation unit 4. This control unit 9 controls, for example, the measuring unit 5 to detect body motion information and biological information. At this point, in the case of detecting and measuring an electrocardiogram of the user with the electrocardiographic measuring unit 54, the control unit 9 causes the impedance measuring unit 55 to carry out the calculation of an impedance value and causes the electrocardiographic measuring unit 54 to carry out the provisional measurement of an electrocardiogram. Then, based on these, the control unit 9 sets one of the electrodes 5411, 5412 of the front surface-side electrode 541 and one of the electrodes 5421, 5422 of the back surface-side electrode 542 as working electrodes, and also sets one of the electrodes that are not set as the working electrodes, as a reference electrode, and then causes the electrocardiographic measuring unit 54 to carry out the measurement of an electrocardiogram (definitive measurement) using these working electrodes and reference electrode.

This control unit 9 has a clocking unit 91, a notification control unit 92, a communication control unit 93, a detection control unit 94, an analysis unit 95, an abnormality determination unit 96, and an electrode setting unit 97, as shown in FIG. 4, as functional units expressed by the processing circuit executing the programs stored in the control information storage unit 81.

### [Configuration of Clocking Unit, Notification Control Unit and Communication Control Unit]

The clocking unit 91 keeps the current time and date.

The notification control unit 92 controls the operation of the notification unit 6. For example, the notification control unit 92 causes the notification unit 6 to notify presentation information including the operation state of the measuring device 1A and a display or sound indicating the result of detection or the like by the measuring unit 5. Also, the notification control unit 92 drives the motor of the vibration unit 63 according to need and thus causes predetermined information to be notified through the vibration generated by the driving of the motor.

The communication control unit 93 controls the operation of the communication unit 7.

### [Configuration of Detection Control Unit]

The detection control unit 94 controls the operation of the measuring unit 5. For example, the detection control unit 94 causes the body motion information detection unit 51 to detect a body motion of the user and also causes the pulse wave detection unit 53 to detect a pulse wave of the user. Then the detection control unit 94 causes the detection information storage unit 82 to store an acceleration signal indicating the body motion and a pulse wave signal indicating the pulse wave, along with the current time and date.

Also, the detection control unit 94 causes the impedance measuring unit 55 to measure the bioelectrical impedance value and also causes the electrocardiographic measuring unit 54 to carry out provisional measurement of an electrocardiogram, under the indication of the electrode setting unit 97, described later. Then, the detection control unit 94 causes the electrocardiographic measuring unit 54 to carry out measurement of an electrocardiogram using working electrodes and a reference electrode set by the electrode setting unit (definitive measurement), and causes the detection information storage unit 82 to store an electrocardiographic signal indicating the measured electrocardiogram, along with the current time and date. Also, the detection control unit 94 may cause the detection information storage unit 82 to store a pulse rate calculated based on the pulse wave signal, as biological information, along with the current time and date.

### [Configuration of Analysis Unit]

The analysis unit 95 analyzes body motion information and biological information inputted from the body motion information detection unit 51 and the biological information detection unit 52.

Specifically, the analysis unit 95 calculates a pulse rate of the user, based on the pulse wave signal inputted from the pulse wave detection unit 53 and the acceleration signal inputted from the body motion information detection unit 51. For example, the analysis unit 95 eliminates a body motion noise component based on the acceleration signal from the pulse wave signal and thus obtains a pulsation signal. The analysis unit 95 then performs frequency analysis such as FFT (fast Fourier transform) on the pulsation signal, extracts a frequency of pulse from the obtained result of the analysis (power spectrum), and calculates the pulse rate based on the frequency of pulse. The analysis unit 95 is not limited to such calculation of the pulse rate and may also calculate the pulse rate by other methods.

Also, the analysis unit 95 generates an R-R waveform signal indicating change with time in the R-R interval (time difference between the R wave of the steepest peak included in the pulse wave signal and the R wave immediately before) for each frame, based on the result of analysis in the frequency analysis. Moreover, the analysis unit 95 calculates a coefficient of variation of R-R interval CVRR and generates a variation coefficient waveform signal indicating change with time in the coefficient of variation of R-R interval CVRR.

Moreover, the analysis unit 95 calculates a pace of walking (pitch) of the user, based on the acceleration signal. For example, the analysis unit 95 performs frequency analysis similar to the above on the acceleration signal, extracts a frequency of body motion from the obtained result of the analysis, and calculates the pace of walking, based on the frequency of body motion.

In addition, the analysis unit 95 analyzes the electrocardiographic signal inputted from the electrocardiographic measuring unit 54.

Then, the analysis unit 95 causes the detection information storage unit 82 to store the calculated pulse rate and pace of walking, and the result of the analysis of the electrocardiogram.

### [Configuration of Abnormality Determination Unit]

The abnormality determination unit 96 determines whether an abnormality categorized as arrhythmias has occurred to the user or not, based on the R-R waveform signal and the variation coefficient waveform signal generated by the analysis unit 95 and the calculated pulse rate. Such arrhythmias may be atrial fibrillation, premature contraction, tachycardia, and bradycardia.

Atrial fibrillation refers to the state where the number of atrial beats is 300 per minute or higher and where the heart beats fast and irregularly, leading to the stagnation of blood in the heart. If atrial fibrillation occurs, the amplitude of the R-R waveform signal increases and the coefficient of variation of R-R interval CVRR changes greatly as well. Therefore, based on these, the abnormality determination unit 96 determines whether atrial fibrillation has occurred or not. However, without being limited to this, the abnormality determination unit 96 may also determine whether atrial fibrillation has occurred or not by other methods. For example, the abnormality determination unit 96 may perform matching between the waveform of the pulse wave signal at the time of occurrence of atrial fibrillation in the past and the waveform of the above pulse wave signal, and determine that atrial fibrillation has occurred if the waveforms are determined as substantially the same.

Premature contraction refers to the state where the heart deviates from its original cycle and contracts earlier due to an abnormal stimulus. If this premature contraction occurs, consequently a different waveform from a waveform of normal sinus rhythm is included in the pulse wave signal. Therefore, the abnormality determination unit 96 performs matching between the waveform at the time of occurrence of premature contraction and the waveform of the acquired pulse wave signal, and determines that premature contraction has occurred if the waveforms are determined as substantially the same. The waveform at the time of occurrence of premature contraction maybe an average waveform or may be the waveform of a premature contraction which occurred to the user in the past.

Tachycardia refers to the state where the pulse is abnormally fast. Bradycardia refers to the state where the pulse is abnormally slow. For example, if it is the state where the pulse of an ordinary adult with a heart rate at rest of 60 to 70 bpm exceeds 100 bpm when the person is not exercising, tachycardia is suspected. If it is the state where the pulse is 50 bpm or below, bradycardia is suspected.

Of these, if tachycardia occurs, the state where the R-R interval is shorter than in normal time continues, and if bradycardia occurs, the state where the R-R interval is longer than in normal time continues. Therefore, if the state where the R-R interval exceeds a threshold of tachycardia that is set according to the user continues for a predetermined time, the abnormality determination unit 96 determines that tachycardia has occurred. Meanwhile, if the state where the R-R interval is below a threshold of bradycardia that is set according to the user (lower threshold than the threshold of tachycardia) continues for a predetermined time, the abnormality determination unit 96 determines that bradycardia has occurred.

If it is determined by such an abnormality determination unit 96 that an abnormality categorized as arrhythmias has occurred to the user, the notification control unit 92 causes the notification unit 6 to notify presentation information which prompts the user to measure an electrocardiogram. For example, the notification control unit 92 causes the display unit 61 to display a message to prompt the measurement of an electrocardiogram. Also, for example, the notification control unit 92 causes the audio output unit 62 to output a predetermined sound (for example, a warning sound), or causes the vibration unit 63 to generate the vibration.

### [Configuration of Electrode Setting Unit]

The electrode setting unit 97 functions when measuring an electrocardiogram of the user. This electrode setting unit 97 selects and sets two working electrodes and a reference electrode (grounding electrode) used for the measurement of an electrocardiogram of the user (definitive measurement) from among the electrodes 5411, 5412 of the front surface-side electrode 541 and the electrodes 5421, 5422 of the back surface-side electrode 542, based on the impedance value measured by the impedance measuring unit 55 and the electrocardiographic waveform of the result of the provisional measurement by the electrocardiographic measuring unit 54.

Specifically, when an operation signal to measure an electrocardiogram of the user is inputted from the operation unit 4, the electrode setting unit 97 causes the impedance measuring unit 55 to measure the impedance value between the electrodes 5411, 5412 and the electrodes 5421, 5422, via the detection control unit 94.

At this time, the electrode setting unit 97 causes the supply electrode switching unit 552 to connect one of the electrodes 5411, 5412 to the current supply unit 551. Also, the electrode setting unit 97 causes the detection electrode switching unit 553 to connect the electrode connected to the current supply unit 551 by the supply electrode switching unit 552 and also one of the electrodes 5421, 5422, to the voltage detection unit 554.

Thus, as shown in FIG. 5, a first path of a current, outputted to the electrode 5411 from the current supply unit 551 via the supply electrode switching unit 552, conducted through the body of the user, and inputted to the voltage detection unit 554 from the electrode 5421 via the detection electrode switching unit 553, is formed. Also, a second path of a current, outputted to the electrode 5411, conducted through the body of the user, and inputted to the voltage detection unit 554 from the electrode 5422 via the detection electrode switching unit 553, is formed.

Moreover, a third path of a current, outputted to the electrode 5412 from the current supply unit 551 via the supply electrode switching unit 552, conducted through the body of the user, and inputted to the voltage detection unit 554 from the electrode 5421 via the detection electrode switching unit 553, is formed. Also, a fourth path of a current, outputted to the electrode 5412, conducted through the body of the user, and inputted to the voltage detection unit 554 from the electrode 5422 via the detection electrode switching unit 553, is formed.

Then, based on the voltage value of the current outputted from the current supply unit 551 and the voltage value of the current conducted through the first path, the impedance value in the first path, that is, the bioelectrical impedance value of the human body between the electrode 5411 and the electrode 5421, is calculated by the impedance calculation unit 555. Similarly, the bioelectrical impedance values in the second path, the third path and the fourth path, that is, the bioelectrical impedance values between the electrode 5411 and the electrode 5422, between the electrode 5412 and the electrode 5421, and between the electrode 5412 and the electrode 5422 are calculated respectively.

The respective impedance values thus calculated are acquired by the electrode setting unit 97.

Next, the electrode setting unit 97 selects and sets two provisional working electrodes and one provisional reference electrode from among the electrodes 5411, 5412 of the front surface-side electrode 541 and the electrodes 5421, 5422 of the back surface-side electrode 542, and causes the electrocardiographic measuring unit 54 to perform provisional measurement of an electrocardiogram via the detection control unit 94.

Specifically, the electrode setting unit 97 causes the front surface-side electrode switching unit 543 of the electrocardiographic measuring unit 54 to set one of the electrodes 5411, 5412 as a provisional working electrode and causes the back surface-side electrode switching unit 544 to set one of the electrodes 5421, 5422 as a provisional working electrode. Moreover, the electrode setting unit 97 causes the reference electrode switching unit 545 to set, as a provisional reference electrode, one of the electrodes that are not set as the provisional working electrodes by the respective switching units 543, 544, from among the respective electrodes 5411, 5412, 5421, 5422.

As such combinations of provisional working electrodes and a provisional reference electrode, there are eight combinations as follows.

A first combination is a combination in which the electrode 5411 and the electrode 5421 are set as provisional working electrodes and in which the electrode 5412 is set as a provisional reference electrode.

A second combination is a combination in which the electrode 5411 and the electrode 5421 are set as provisional working electrodes and in which the electrode 5422 is set as a provisional reference electrode.

A third combination is a combination in which the electrode 5411 and the electrode 5422 are set as provisional working electrodes and in which the electrode 5412 is set as a provisional reference electrode.

A fourth combination is a combination in which the electrode 5411 and the electrode 5422 are set as provisional working electrodes and in which the electrode 5421 is set as a provisional reference electrode.

A fifth combination is a combination in which the electrode 5412 and the electrode 5421 are set as provisional working electrodes and in which the electrode 5411 is set as a provisional reference electrode.

A sixth combination is a combination in which the electrode 5412 and the electrode 5421 are set as provisional working electrodes and in which the electrode 5422 is set as a provisional reference electrode.

A seventh combination is a combination in which the electrode 5412 and the electrode 5422 are set as provisional working electrodes and in which the electrode 5411 is set as a provisional reference electrode.

An eighth combination is a combination in which the electrode 5412 and the electrode 5422 are set as provisional working electrodes and in which the electrode 5421 is set as a provisional reference electrode.

The electrode setting unit 97 then causes the electrocardiographic measuring unit 54 to perform the provisional measurement of an electrocardiogram with each of these combinations under the control of the detection control unit 94.

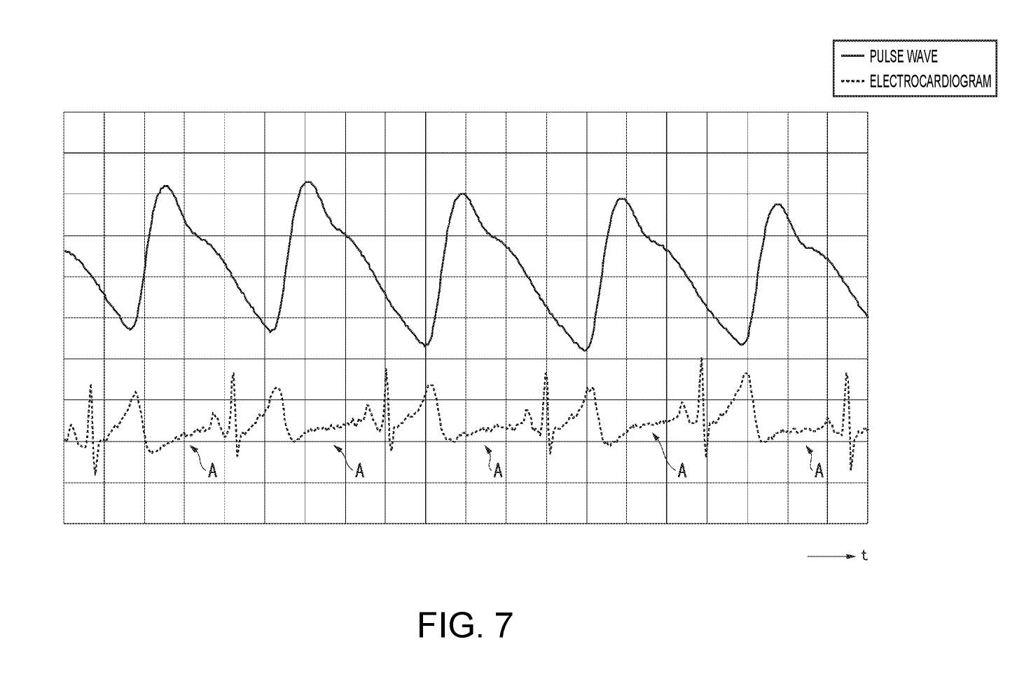

FIG. 7 is a view showing an example of waveforms of the measured pulse wave and electrocardiogram.

Here, for example, if there is an influence of an electromagnetic induction noise or the like from utility power, a noise is included in the detected electrocardiogram and the waveform of the electrocardiogram becomes irregular. Also, if the grounding is not good, the base line (part indicated by arrow A) of the electrocardiographic waveform shown in FIG. 7 becomes irregular or fluctuates. In such cases, it is difficult to accurately measure and record an electrocardiographic waveform.

To cope with this, the electrode setting unit 97 sets a combination of provisional working electrodes and a provisional reference electrode in which the bioelectrical impedance value between the provisional working electrodes is low and in which the detected electrocardiographic waveform (particularly the base line of the electrocardiographic waveform) has less irregularity, fluctuation and noise, from among the first to eighth combinations, as the working electrodes and the reference electrode used for the definitive measurement of an electrocardiogram.

When a combination of two working electrodes and one reference electrode is thus set, the detection control unit 94 causes the electrocardiographic measuring unit 54 to perform the definitive measurement of an electrocardiogram using these electrodes, and causes the detection information storage unit 82 to store an electrocardiographic signal inputted from the electrocardiographic measuring unit 54.

### [Electrocardiographic Measurement Processing]

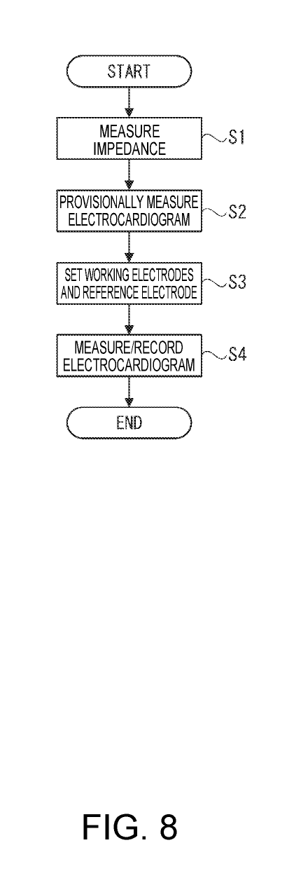

FIG. 8 is a flowchart showing electrocardiographic measurement processing.

In the measuring device 1A, for example, the presentation information which prompts the measurement of an electrocardiogram is presented or the like, and an input operation to measure an electrocardiogram is carried out by the user, as described above. When an operation signal corresponding to the input operation is inputted to the control unit 9 from the operation unit 4, the control unit 9 reads out the electrocardiographic measurement program and executes the electrocardiographic measurement processing described below.

In this electrocardiographic measurement processing, first, the electrode setting unit 97 causes the impedance measuring unit 55 to measure the impedance values in the first to fourth paths via the detection control unit 94 (Step S1), as shown in FIG. 8.

After that, the electrode setting unit 97 causes the electrocardiographic measuring unit 54 to perform the provisional measurement of an electrocardiogram of the user with each of the first to eighth combinations via the detection control unit 94 (Step S2).

The order of execution of these Steps 51, S2 may be reversed.

Next, the electrode setting unit 97 sets one of the electrodes 5411, 5412 and one of the electrodes 5421, 5411 as working electrodes and sets one of the electrodes that are not set as these working electrodes, as a reference electrode, based on the bioelectrical impedance values in the respective paths measured in the above Step 51 and the electrocardiographic waveforms with the respective combinations provisionally measured in the above Step S2 (Step S3).

Then, the detection control unit 94 causes the electrocardiographic measuring unit 54 to measure an electrocardiogram of the user (definitive measurement) using the two working electrodes and the one reference electrode that are set, and causes the detection information storage unit 82 to store an electrocardiographic signal inputted from the electrocardiographic measuring unit 54 (Step S4).

Such definitive measurement of an electrocardiogram is carried out for a predetermined time. As the predetermined time passes, the notification control unit 92 causes the notification unit 6 to notify presentation information that the measurement of the electrocardiogram is finished, and then the electrocardiographic measurement processing ends.

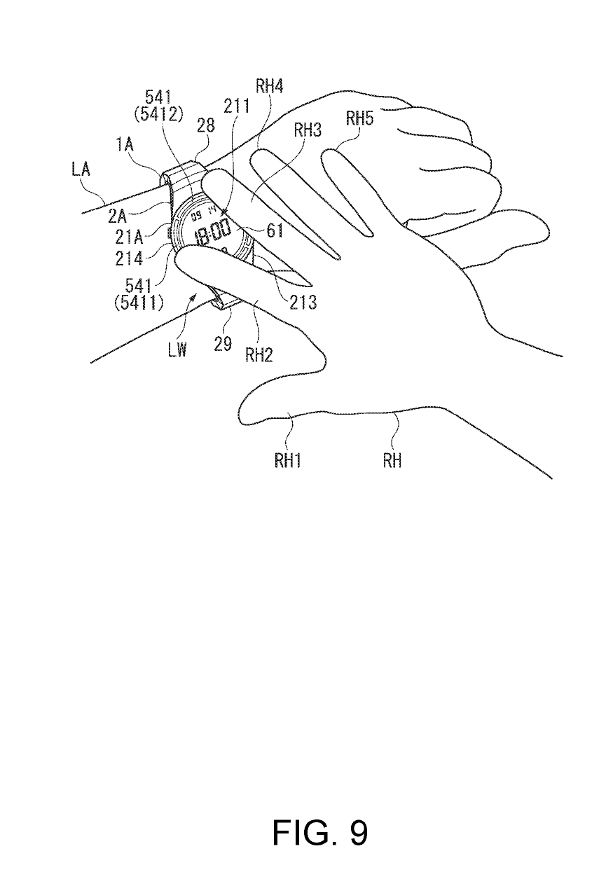

FIG. 9 is a schematic view showing the state where a forefinger RH2 and a middle finger RH3 of a right hand RH of a user wearing the measuring device 1A on a wrist (left wrist) LW of a left arm LA are in contact with the electrodes 5411, 5412.

When the user wears the measuring device 1A on the left wrist LW, the electrodes 5421, 5422 of the back surface-side electrode 542 are in contact with the skin of the left wrist LW. In this wearing state, the user can contact at least one of the electrodes 5411, 5412 of the front surface-side electrode 541 with the right hand RH, as shown in FIG. 9.

In such a wearing state, if the user places the right hand RH on each of the electrodes 5411, 5412, the electrode setting unit 97 sets two working electrodes and one reference electrode, based on the results of the measurement of the bioelectrical impedance in the first to fourth paths and the result of the provisional measurement of an electrocardiogram with the first to eighth combinations, as described above.

Meanwhile, if only one of the electrodes 5411, 5412 is in contact with the right hand RH, the impedance in the path through which the current is conducted between the other electrode and one of the electrodes 5421, 5422 of the back surface-side electrode 542 does not change.

For example, in the above wearing state, if the right hand RH is in contact with the electrode 5411, the impedance values in the first path and the second path are lower than the impedance values in the case where the user is not in contact with the electrode 5411. However, in this case, the impedance values in the third path and the fourth path do not change, compared with the impedance values in the case where the user is not in contact with the electrode 5412.

Therefore, the electrode setting unit 97 can grasp the electrode which the user is in contact with and the electrode which the user is not in contact with, of the electrodes 5411, 5412, based on the changes in the impedance values in the first to fourth paths.

Then, if there is an electrode which the user is not in contact with, of the front surface-side electrode, the electrode setting unit 97 causes the provisional measurement of an electrocardiogram to be carried out with a combination in which this electrode is not set as the provisional working electrode, from among the first to eighth combinations.

For example, if it is determined that the electrode 5412 is not in contact with the user, the electrode setting unit 97 causes the electrocardiographic measuring unit 54 to carry out the provisional measurement of an electrocardiogram with the first to fourth combinations from among the first to eighth combinations.

Thus, the time for the provisional measurement of an electrocardiogram can be reduced and hence the time until the end of the measurement of an electrocardiogram can be reduced.

### [Arrangement of Front Surface-Side Electrode]

In the state where the measuring device 1A is worn on the left wrist LW, the user is considered to touch the electrodes 5411, 5412 with the right hand RH, as shown in FIG. 9. In this case, it is conceivable that the user tries an electrical contact between the electrodes 5411, 5412 and the human body by placing the palm of the right hand RH to cover the front surface 211. Other than this, it is conceivable that the user tries an electrical contact between the respective electrodes 5411, 5412 and the human body by placing two fingers of a thumb RH1, the forefinger RH2, the middle finger RH3, a third finger RH4 and a little finger RH5 of the right hand RH on the electrodes 5411, 5412. In any of these cases, the right hand RH of the user is arranged in such a way as to intersect with the left arm LA at a predetermined angle of intersection (for example, 50 degrees), as viewed from the top side to the user.

In such an arrangement state of the right hand RH, the electrodes 5411, 5412 are arranged at positions where the fingers RH1 to RH5 of the right hand RH can be easily placed.

In the description below, a direction which passes through the center C1 of the front surface 211, as viewed from the position directly opposite the front surface 211 of the main body part 21A, and in which the strap 28 extends from the main body part 21A, is defined as a Y-direction. A direction orthogonal to the Y-direction and heading from the left to the right is defined as an X-direction. In other words, the Y-direction is a direction orthogonal to the direction of the normal line to the display surface of the display unit 61 and along the direction in which the strap 28 extends from the main body part 21A. The X-direction is a direction orthogonal to the direction of the normal line and the Y-direction.

Of the electrodes 5411, 5412 of the front surface-side electrode 541, the electrode 5411 is arranged mainly at the lower left and the electrode 5412 is arranged mainly at the upper right, as viewed from the position directly opposite the front surface 211.

Dividing positions DP1, DP2 of these electrodes 5411, 5412 are situated on a straight line L1 which intersects with the X-direction and the Y-direction at a predetermined angle of intersection (for example, 45 degrees) and which passes through the center C1, the upper left and the lower right of the front surface 211. That is, if the substantially circular display unit 61 arranged on the main body part 21A is seen as the face of an analog clock, the dividing positions DP1, DP2 are situated in the direction of the hour hand indicating half past four and in the direction of the hour hand indicating half past ten.

Here, it is assumed that the dividing positions of the electrodes 5411, 5412 are situated on a straight line which passes through the center C1 and is parallel to the Y-direction, and that the electrodes 5411, 5412 are situated on the left-hand side and the right-hand side, as viewed from the position directly opposite the front surface 211. That is, it is assumed that the dividing positions of the electrodes 5411, 5412 are situated in the direction of 12 o'clock and the direction of 6 o'clock. In this case, for example, if the forefinger RH2 and the middle finger RH3 of the right hand RH are to come in contact with the respective electrodes 5411, 5412, the right hand RH needs to be substantially orthogonal to the left arm LA. An attitude in which the right hand RH is arranged in this manner is a difficult attitude for the human body to take. Therefore, with such arrangement of the electrodes 5411, 5412, it is difficult for the user to place fingers independently on the respective electrodes 5411, 5412.

In contrast, in this embodiment, since the electrodes 5411, 5412 are separated from each other at the dividing positions DP1, DP2 on the straight line L1, it can be made easier to place the fingers RH1 to RH5 independently on each of these electrodes 5411, 5412. Thus, since contact with the front surface-side electrode 541 can be made easier, proper working electrodes and reference electrode can be selected according to the electrocardiographic measurement processing, and hence an electrocardiogram can be measured properly.

### Effects of First Embodiment

The measuring device 1A according to the embodiment described above has the following effects.

By the electrode setting unit 97, one of the electrodes 5411, 5412 forming the front surface-side electrode 541 and one of the electrodes 5421, 5422 forming the back surface-side electrode 542 are set as working electrodes, and one of the electrodes that are not set as the working electrodes is set as a reference electrode. Thus, not only working electrodes suitable for the measurement of an electrocardiogram can be set but also a proper electrode can be set as a reference electrode. Therefore, since the electrocardiographic detection unit 547 detects an electrocardiogram of the user, using these working electrodes and reference electrode, the detection accuracy of the electrocardiogram can be improved and the electrocardiogram can be detected and measured accurately.

The reference electrode is set, based on the impedance value between one of the electrodes 5411, 5412 of the front surface-side electrode 541 which can come into contact with the user and one of the electrodes 5421, 5422 of the back surface-side electrode 542 which comes into contact with the user when the measuring device 1A is worn. Thus, since the reference electrode can be set based on the impedance values (bioelectrical impedance values) detected in the first to fourth paths, a proper reference electrode can be set. Thus, the detection accuracy of the electrocardiogram can be improved further and the electrocardiogram can be detected and measured more accurately.

Here, irregularities occur in the electrocardiographic waveform (particularly the base line), not only in the case where a proper reference electrode is not selected but also in the case where, for example, there is an influence of an electromagnetic induction noise or the like.

To cope with this, the electrode setting unit 97 sets working electrodes and a reference electrode used in the definitive measurement of an electrocardiogram, based on the waveform of an electrocardiographic signal detected with each combination of provisional working electrodes and a provisional reference electrode, and a noise included in the electrocardiographic signal. Thus, proper working electrodes and reference electrode can be selected and set, based on an electrocardiogram that is actually detected. Therefore, the detection accuracy of the electrocardiogram can be improved further and the electrocardiogram can be measured more accurately.

Since the back surface 212 is a surface on the side of the wearing site (for example, the left wrist LW) of the main body part 21A when the measuring device 1A is worn by the user, the respective electrodes 5421, 5422 of the back surface-side electrode 542 can be securely brought into contact with the human body of the user. Also, since front surface 211, where the front surface-side electrode 541 is arranged, is a surface opposite to the back surface 212, it can be made easier to bring the human body (for example, the right hand RH) of the user into contact with the respective electrodes 5411, 5412 of the front surface-side electrode 541 by placing a hand or the like. Therefore, the detection and measurement of an electrocardiogram of the user can be carried out in a simple manner. Also, the electrically continuous path between the front surface-side electrode 541 and the back surface-side electrode 542 can be made long and the detection accuracy of the electrocardiogram can be improved.

Since the electrodes 5411, 5412 of the front surface-side electrode 541 are arranged in the electrode arrangement part 23 surrounding the display unit 61 arranged on the front surface 211, the plurality of electrodes 5411, 5412 can be arranged without limiting the arrangement of the display unit 61. Also, with such an arrangement, the respective electrodes 5411, 5412 can be each formed in an arc-shape along the electrode arrangement part 23 and can be arranged separately at the lower left and upper right sites of the front surface 211. Therefore, the respective electrodes 5411, 5412 can be brought into contact with the user, electrically independently of each other. Moreover, with the display unit 61, it is possible to present biological information such as the detected electrocardiogram to the user.

The electrodes 5411, 5412 provided in the front surface-side electrode 541 are separated from each other at the dividing positions DP1, DP2 on the straight line L1 intersecting with the X-direction and the Y-direction at substantially 45 degrees. The electrodes 5411, 5412 are arranged respectively at the lower left and upper right sites of the front surface 211. That is, if the display unit 61 is seen as the face of an analog clock, the respective electrodes 5411, 5412 are separated at the position of the hour hand indicating half past four and the position of the hour hand indicating half past ten. Thus, for example, when the measuring device 1A is worn on the left wrist LW, it can be made easier to bring the fingers RH1 to RH5 of the right hand RH into contact with each of these electrodes 5411, 5412 independently. Therefore, it can be made easier to carry out the detection and measurement of an electrocardiogram.

The measuring device 1A has the pulse wave detection unit 53, which detects a pulse wave of the user, in addition to the electrocardiographic measuring unit 54 and the impedance measuring unit 55. Thus, since biological information of the user that is detected can be increased, the versatility and convenience of the measuring device 1A can be improved.

### Modifications of First Embodiment

In the measuring device 1A, the casing 2A is formed in the shape of a wristwatch, and the main body part 21A forming the casing 2A is formed in a substantially circular shape. However, the main body part is not limited to this and may be formed in a substantially rectangular shape.

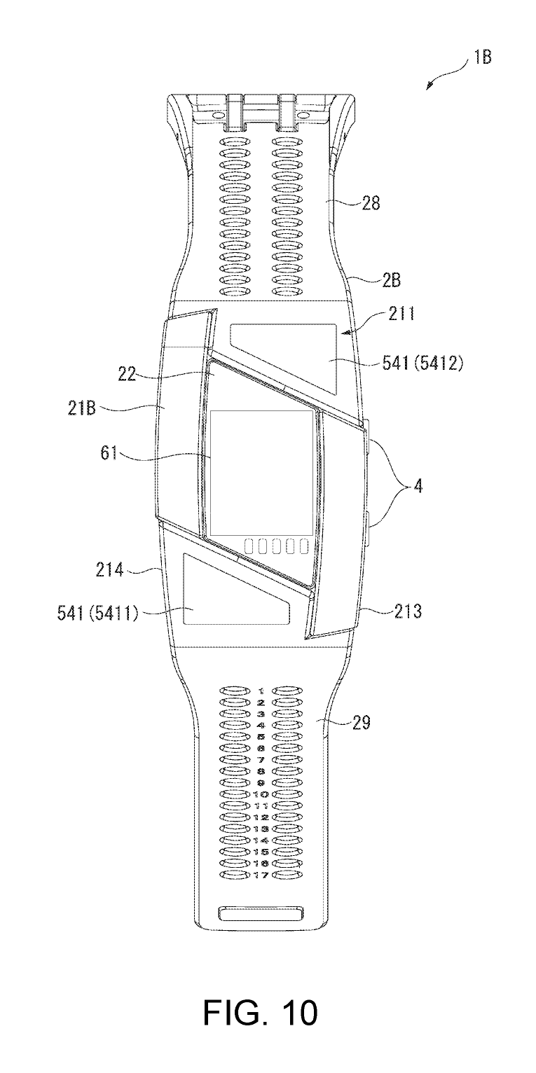

FIG. 10 is a front view showing a biological information measuring device 1B, which is a modification of the measuring device 1A.

For example, the biological information measuring device 1B has a configuration and functions similar to those of the measuring device 1A, except for having a casing 2B instead of the casing 2A, as shown in FIG. 10. Also, the casing 2B has a configuration similar to that of the casing 2A, except for having a main body part 21B instead of the main body part 21A.

The main body part 21B is formed in a substantially rectangular shape with its longitudinal direction being the direction in which the straps 28, 29 extend from the main body part 21B (the above Y-direction). A substantially rectangular display unit 61 is arranged substantially at the center of the front surface 211 of this main body part 21B. In the measuring device 1B, the casing 2B in which the main body part 21B and the straps 28, 29 are integrated is employed.

In the measuring device 1B having such a main body part 21B, the electrodes 5411, 5412 of the front surface-side electrode 541 are arranged, insulated from each other, at lower left and upper right positions on the front surface 211, as viewed from a position directly opposite the front surface 211 of the measuring device 1B worn by the user. In other words, the electrodes 5411, 5412 are arranged at positions on the opposite sides of the display unit 61 on the front surface 211 (positions on one end side and the other end side in the above Y-direction).

With such an arrangement of the electrodes 5411, 5412, too, it can be made easier to place the right hand RH (particularly the fingers RH1 to RH5) on the electrodes 5411, 5412 of the measuring device 1B worn on the left wrist LW of the user.

The measuring device 1B described above, too, can achieve effects similar to those of the measuring device 1A.

### Second Embodiment

Next, a second embodiment of the invention will be described.

A biological information measuring device according to this embodiment has a configuration similar to those of the biological information measuring devices 1A, 1B but differs from the biological information measuring devices 1A, 1B in that the number of electrodes provided in the front surface-side electrode is different. In the description below, the same parts or substantially the same parts as already described parts are denoted by the same reference numbers and the description thereof is omitted.

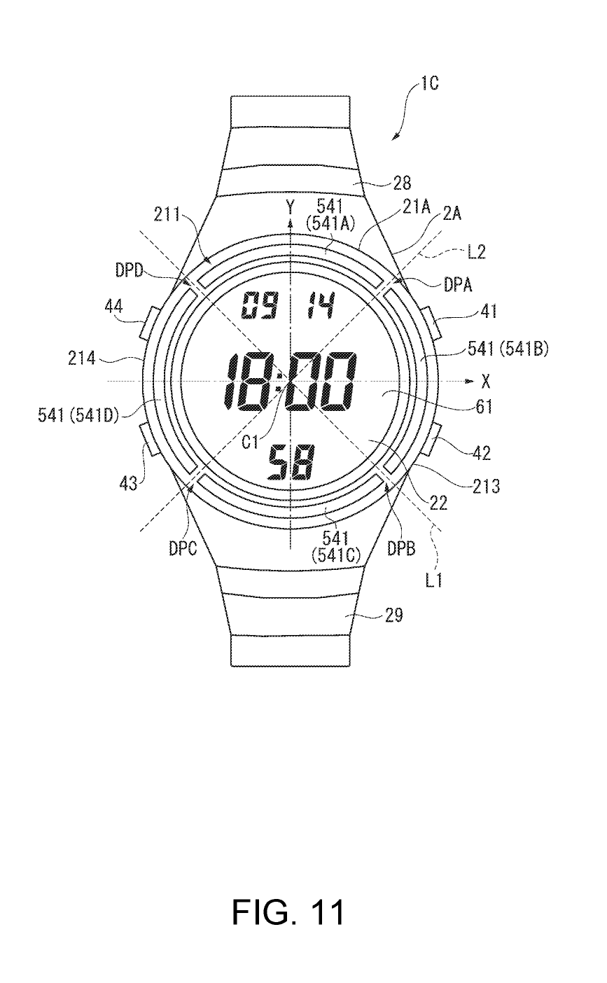

FIG. 11 is a front view showing a biological information measuring device 1C according to this embodiment.

The biological information measuring device 1C according to this embodiment has a configuration and functions similar to those of the measuring device 1A, except that the front surface-side electrode 541 has four electrodes 541A, 541B, 541C, 541D, as shown in FIG. 11.

The electrodes 541A to 541D are arranged on the front surface 211 of the main body part 21A, similarly to the electrodes 5411, 5412. When described in detail, the electrodes 541A to 541D are arranged electrically independently of each other in the electrode arrangement part 23 formed around the display unit 61.

When described in detail, the electrodes 541A to 541D are separated and insulated from each other at four dividing positions DPA to DPD. Of these dividing positions DPA to DPD, the dividing positions DPB, DPD are situated on the straight line L1. Meanwhile, the dividing positions DPA, DPC are situated on a straight line L2 orthogonal to the straight line L1 as viewed from a position directly opposite the front surface 211 (straight line L2 intersecting with each of the X-direction and the Y-direction at an angle of intersection of about 45 degrees and passing through the center C1, the lower left and the upper right of the front surface 211). That is, if the display unit 61 is seen as the face of an analog clock, the dividing positions DPA to DPD are situated in the directions of the hour hand indicating half past one, half past four, half past seven, and half past ten, respectively. Therefore, the electrodes 541A to 541D are situated on the upper side, the right-hand side, the lower side, and the left-hand side on the front surface 211, respectively.

In the electrocardiographic measuring unit 54 having such electrodes 541A to 541D, the front surface-side electrode switching unit 543 is connected to the electrodes 541A to 541D, and the reference electrode switching unit 545 is connected to these electrodes 541A to 541D and the electrodes 5421, 5422 of the back surface-side electrode 542.

Also, the supply electrode switching unit 552 of the impedance measuring unit 55 is connected to the electrodes 541A to 541D. The detection electrode switching unit 553 is connected to these electrodes 541A to 541D and the electrodes 5421, 5422.

Also, the electrode setting unit 97 causes the impedance measuring unit 55 to measure the impedance values in all of the paths connecting one of the electrodes 541A to 541D and one of the electrodes 5421, 5422, via the detection control unit 94.

Moreover, the electrode setting unit 97 causes the electrocardiographic measuring unit 54 to carry out provisional measurement of an electrocardiogram with all combinations in which one of the electrodes 541A to 541D and one of the electrodes 5421, 5422 are set as provisional working electrodes and in which one of the electrodes that are not set as these provisional working electrodes is set as a provisional reference electrode, via the detection control unit 94.

After that, the electrode setting unit 97 sets one of the electrodes 541A to 541D and one of the electrodes 5421, 5422 as working electrodes and sets one of the electrodes that are not set as these working electrodes, as a reference electrode, based on the results of the measurement of the impedance values and the result of the provisional measurement of the electrocardiogram.

Then, the detection control unit 94 causes the electrocardiographic measuring unit 54 to measure an electrocardiogram of the user, using the two working electrodes and the one reference electrode that are set, and causes the detection information storage unit 82 to store the resulting electrocardiographic signal.

The measuring device 1C according to this embodiment described above can achieve effects similar to those of the measuring device 1A.

### Third Embodiment

Next, a third embodiment of the invention will be described.

A biological information measuring device according to this embodiment has a configuration similar to those of the biological information measuring devices 1A to 1C but differs from the biological information measuring devices 1A to 1C in that the arrangement of the back surface-side electrode is different. In the description below, the same parts or substantially the same parts as already described parts are denoted by the same reference numbers and the description thereof is omitted.

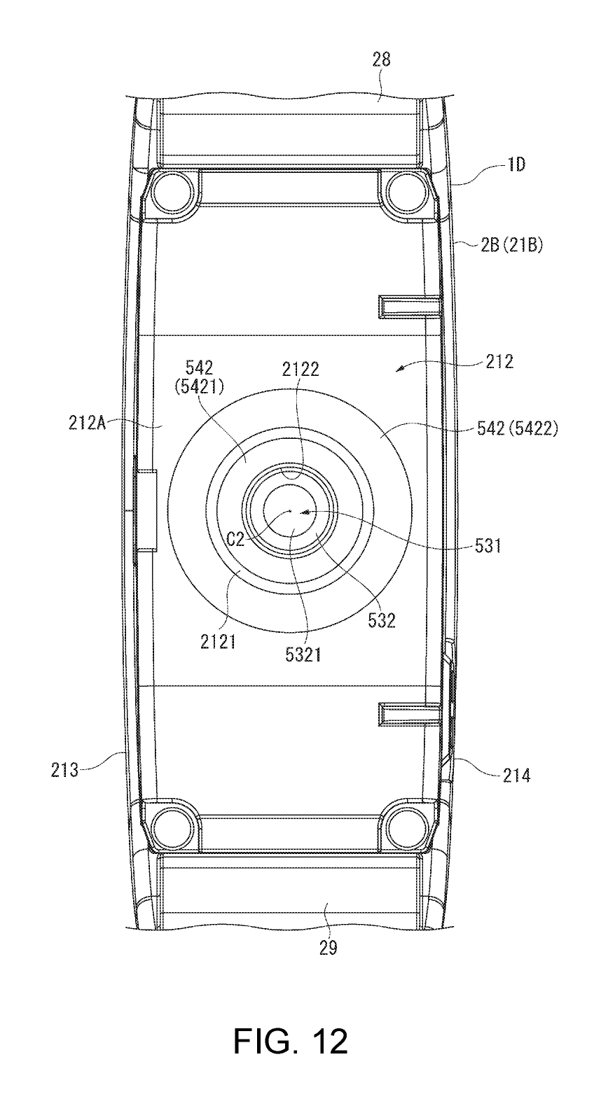

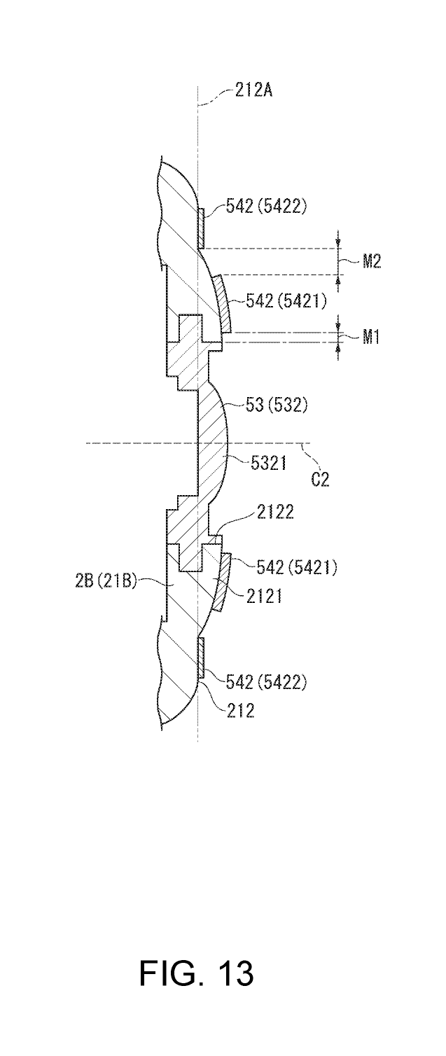

FIG. 12 is a back view showing a biological information measuring device 1D according to this embodiment. Also, FIG. 13 is a cross-sectional view showing a site on the side of the back surface 212 of the main body part 21B, and a light-transmitting member 532 forming the pulse wave sensor 531. FIG. 13 is a cross-sectional view in a direction of connecting the front surface 211 and the back surface 212.

The biological information measuring device 1D has a configuration similar to that of the biological information measuring device 1B, except that the configuration of the back surface 212 and the arrangement of the back surface-side electrode 542 are different.

In this measuring device 1D, a protruding part 2121 is formed on the back surface 212 of the main body part 21B forming the casing 2B, as shown in FIG. 12. This protruding part 2121 is formed in a gently convex curved surface so that the amount of protrusion from a reference surface 212A of the back surface 212 (plane connecting the corners of the back surface 212) increases as it goes toward the center C2 from the outer edge side of the back surface 212, as shown in FIG. 13. That is, the protruding part 2121 protrudes from the reference surface 212A more largely at a position close to the center C2 than at a position on the side of the outer edge of the back surface 212.

At the center of the protruding part 2121, a detection window 2122 which is a circular opening is formed, as shown in FIG. 12 and FIG. 13. The light-transmitting member 532 forming the pulse wave detection unit 53 is fitted in this detection window 2122, and the light-transmitting member 532 covers the light emitting element and the light receiving element (not illustrated) of the pulse wave sensor 531 provided inside the main body part 21B. That is, the protruding part 2121 also functions as a light shielding part which prevents light from a site other than the detection window 2122 from becoming incident on the light receiving element of the pulse wave sensor 531.

Also, a swelling part 5321 swelling in an arc-shape is formed substantially at the center of the light-transmitting member 532. If the reference surface 212A is used as the point of reference, the height position of the swelling part 5321 is higher than the height position of an end part close to the center C2 which is the most protruding site of the protruding part 2121. That is, the apex of the swelling part 5321 is further away from the reference surface 212A than the protruding part 2121.

Of the back surface-side electrode 542 arranged on such a back surface 212, the electrode 5421 is arranged in a ring-shape at a site on the side of the detection window 2122 of the protruding part 2121, and the electrodes 5422 is arranged in a ring-shape at a site on the outer side of the protruding part 2121.

Of these, the electrode 5421 is arranged at a position closer to the center C2 than a position on the side of the outer edge in the protruding part 2121. In other words, the electrode 5421 is arranged on the protruding part 2121 in such a way that a dimension Ml between the electrode 5421 and the edge of the detection window 2122 is smaller than a dimension M2 between the electrode 5421 and the outer edge of the protruding part 2121. The height position of this electrode 5421 from the reference surface 212A is higher than the height position of the swelling part 5321. When described in detail, the electrode 5421 is arranged at the most distant position from the reference surface 212A, of the components situated on the back surface 212.

The measuring device 1D according to this embodiment described above can achieve effects similar to those of the measuring devices 1A to 1C and can also achieve the following effects.

Since the electrode 5421 of the back surface-side electrode 542 is arranged on the protruding part 2121, the electrode 5421 can be brought into tight contact with the wearing site, when the measuring device 1D is worn at the wearing site and the swelling part 5321 comes into tight contact with the wearing site. Therefore, an electrocardiogram can be detected accurately.

In the measuring device 1D, the back surface-side electrode 542 has the electrodes 5421, 5422. However, the back surface-side electrode 542 may be configured to have the electrode 5421 only, or may be configured to have another electrode in addition to the electrodes 5421, 5422. Moreover, each electrode forming the back surface-side electrode 542 is not limited to the ring-shape and may be divided into a plurality of electrodes.

Also, the configuration of the measuring device 1D may be such that the measuring device 1D has the casing 2A having the main body part 21A formed in a substantially circular shape as viewed from the back side, instead of the casing 2B having the main body part 21B which is substantially rectangular as viewed from the back side, and the above configuration may be applied to the back surface 212 of the main body part 21A.

### Fourth Embodiment

Next, a fourth embodiment of the invention will be described.

A biological information measuring device according to this embodiment has a configuration similar to those of the biological information measuring devices 1A to 1D. Here, in the measuring devices 1A to 1D, the light emitting element and the light receiving element forming the pulse wave sensor 531 are covered with the light-transmitting member 532, and the back surface-side electrode 542 is arranged in the casings 2A, 2B. In contrast, in the measuring device according to this embodiment, the light emitting element and the light receiving element are arranged on the back surface 212, and the back surface-side electrode 542 is provided on the pulse wave sensor 531. In this respect, the measuring device according to this embodiment differs from the measuring devices 1A to 1D. In the description below, the same parts or substantially the same parts as already described parts are denoted by the same reference numbers and the description thereof is omitted.

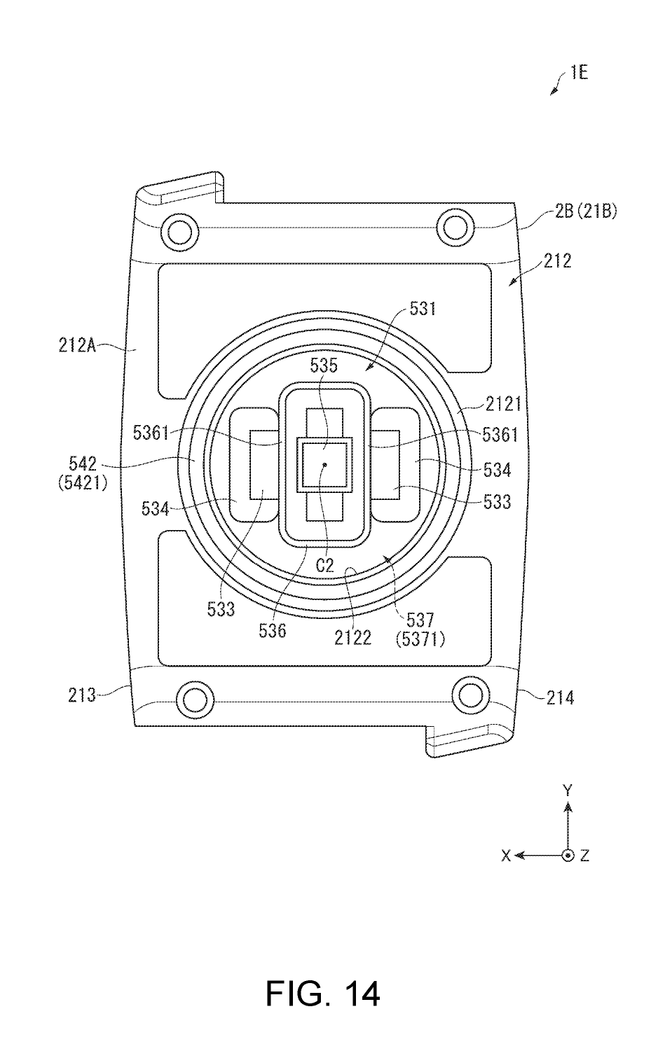

FIG. 14 is a back view showing a biological information measuring device 1E according to this embodiment. In FIG. 14, the illustration of the straps 28, 29 is omitted.

The biological information measuring device 1E according to this embodiment has a configuration similar to that of the measuring device 1D, except that the configuration and arrangement of the pulse wave sensor 531 and the configuration and arrangement of the back surface-side electrode 542 are different.

In this measuring device 1E, the pulse wave sensor 531 is arranged substantially at the center of the protruding part 2121 on the back surface 212, as shown in FIG. 14. However, the pulse wave sensor 531 is not provided with the light-transmitting member 532. That is, in the measuring device 1E, a substrate 537 forming the pulse wave sensor 531 is arranged at a position that is on the inner side of the protruding part 2121 as viewed from a position facing the back surface 212 and to the side of the front surface 211 from the protruding part 2121. In other words, the substrate 537 is arranged in such a way as to abut against the surface opposite to the contact surface that comes into contact with the human body at a site (back surface part) of the main body part 21B forming the back surface 212 (second surface). Also, a light emitting element 533, a reflection part 534, a light receiving element 535 and a light shielding wall 536, similarly forming the pulse wave sensor 531 and arranged on the substrate 537, are exposed outside within the arrangement range of the protruding part 2121 as viewed from a position facing the back surface 212. That is, in this embodiment, the circular detection window 2122 is not arranged in the protruding part 2121. It can be said that the part where the light emitting element 533, the reflection part 534, the light receiving element 535 and the light shielding wall 536 are exposed, in the protruding part 2121, is a detection window.

In the description below, of directions along a normal line to amounting surface 5371 where the light emitting element 533 or the like is mounted on the substrate 537, the direction toward the back surface 212 from the front surface 211 of the main body part 21B is defined as a Z-direction. The direction which is orthogonal to the Z-direction and in which the strap 28 extends from the main body part 21B (upper direction as viewed in the illustration of FIG. 14) is defined as a Y-direction. Of directions orthogonal to each of the Y-direction and the Z-direction, the direction toward the right lateral surface 213 from the left lateral surface 214 (left direction as viewed in the illustration of FIG. 14) is defined as an X-direction.

Hereinafter, the pulse wave sensor 531 will be described in detail.

The substrate 537 electrically connected to the control unit 9 is arranged on the inner side of the protruding part 2121 (at a site opposite to the Z-direction to the protruding part 2121 within the arrangement range of the protruding part 2121 as viewed from the Z-direction side), in such a way that the mounting surface 5371 of the substrate 537 faces the inner surface of the protruding part 2121. On this mounting surface 5371, a pair of light emitting elements 533, a pair of reflection parts 534, the light receiving element 535, and the light shielding wall 536 are arranged.

The pair of light emitting elements 533 is arranged respectively at positions on the both end sides (the side of the right lateral surface 213 and the side of the left lateral surface 214) in the X-direction on the substrate 537. That is, the respective light emitting elements 533 are arranged along the X-direction.

Moreover, the reflection parts 534 which reflect the light incident from the respective light emitting elements 533 toward the wearing site are provided at positions on the outer side from the respective light emitting elements 533, on the substrate 537. In other words, the pair of reflection parts 534 is arranged in such a way as to sandwich the pair of light emitting elements 533 in the X-direction. Each light emitting element 533 is made up of an LED, as described above.

The single light receiving element 535 is arranged at a position which is sandwiched by these light emitting elements 533 and which is substantially at the center of the substrate 537 (position corresponding to the center C2). This light receiving element 535 is made up of a photodiode, as described above.

Around the light receiving element 535, the light shielding wall 536, which is substantially rectangular as viewed from a position facing the back surface 212, stands up from the substrate 537. This light shielding wall 536 is to prevent the light emitted from each light emitting element 533 from becoming incident directly on the light receiving element 535 without traveling via the wearing site. A sidewall part 5361 along the long side of the light shielding wall 536 (sidewall part 5361 along the Y-direction) is situated between each light emitting element 533 and the light receiving element 535.

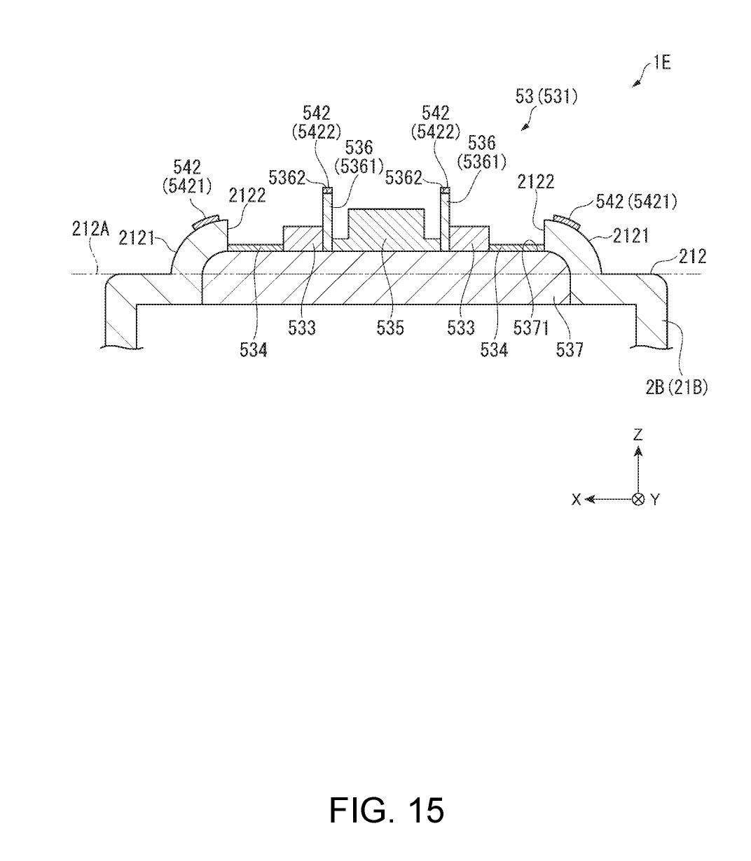

FIG. 15 is a cross-sectional view showing the configuration on the side of the back surface 212 in the measuring device 1E. Also, FIG. 15 is a cross-sectional view in a direction of connecting the front surface 211 and the back surface 212, and a cross-sectional view on an XZ-plane passing through the center of the light receiving element 535.

The height position of such a light shielding wall 536 from the substrate 537 (the most distant position from the substrate 537) is higher than the height positions of the light receiving element 535 and the light emitting elements 533, as shown in FIG. 15. With such a configuration, the light from the light emitting elements 533 can be restrained from becoming incident directly on the light receiving element 535 without traveling via the human body.

Also, the height position of the light receiving element 535 is higher than the height position of the light emitting elements 533. That is, if the mounting surface 5371 of the substrate 537 is used as the point of reference, the height position of the light receiving element 535 is higher than the height position of the light emitting elements 533, and the height position of the light shielding wall 536 is higher than the height position of the light receiving element 535. With such a configuration, the light receiving element 535 is arranged at a position close to the human body. Therefore, it can be made easier for the light receiving element 535 to detect the light emitted from the light emitting elements 533 and traveling through the human body.

Meanwhile, if the reference surface 212A is used as the point of reference, the height position of the protruding part 2121 is higher than the height position of the light emitting elements 533 and lower than the height position of the light receiving element 535. Similarly, the height position of the light shielding wall 536 is higher than the height position of the protruding part 2121. That is, the most protruding component from the reference surface 212A (the component whose end part on the Z-direction side is the most distant from the reference surface 212A) on the back surface 212 is the light shielding wall 536. Using the mounting surface 5371 of the substrate 537 as a reference surface results in a similar arrangement. With such a configuration, the pulse wave sensor 531 can be stably brought into contact with the human body and a pulse wave can be detected stably.

Also, in this embodiment, of the electrodes 5421, 5422 forming the back surface-side electrode 542, the electrode 5421 is arranged on the protruding part 2121, as in the case of the measuring device 1D. The position of the electrode 5421 on the protruding part 2121 is similar to that in the measuring device 1D.

Meanwhile, the electrode 5422 is arranged on an end surface 5362 on the side of the direction of protrusion from the substrate 537, of the light shielding wall 536 (surface substantially parallel to the substrate 537 and the reference surface 212A, of the light shielding wall 536). With such a configuration, a plurality of electrodes which stably comes into contact with the human body can be arranged on the surface of the back surface 212, and the biological information measuring device 1E having both a detection ability for pulse wave signals and a detection ability for electrocardiographic signals can be formed.

In FIG. 15 described above, the light emitting elements 533, the reflection part 534 and the light receiving element 535 are arranged, exposed in the space within the detection window 2122. However, in order to protect these components, these components maybe covered with a transparent member. For example, as shown in FIG. 15, the space around the light emitting elements 533, the reflection part 534 and the light receiving element 535 may be filled with a member which transmits light corresponding to the sensitivity zone of the light receiving element 535, such as an epoxy resin or polycarbonate resin. Thus, the environmental tolerance and physical strength of the pulse wave sensor 531 can be secured.

Also, the pair of light emitting elements 533 is arranged in such a way as to sandwich the light receiving element 535 in the X-direction. However, the pair of light emitting elements 533 may be arranged in such a way as to sandwich the light receiving element 535 in the Y-direction. That is, the arrangement of the pulse wave sensor 531 may be rotated about the center C2 on the XY-plane by 90 degrees from the foregoing arrangement, or may be rotated by a predetermined angle.

The measuring device 1E according to this embodiment described above can achieve effects similar to those of the measuring device 1D and can also achieve the following effects.

Of the electrodes 5421, 5422 provided in the back surface-side electrode 542, the electrode 5421 is arranged on the end surface 5362 of the light shielding wall 536, which protrudes the most from the reference surface 212A on the back surface 212. Thus, when the measuring device 1E is worn at the wearing site on the user, the electrode 5421 can be securely brought into contact with the wearing site. Therefore, an electrocardiogram of the user can be detected and measured more accurately.

In the measuring device 1E, the back surface-side electrode 542 has the two electrodes 5421, 5422. However, the back surface-side electrode 542 may be configured to be provided only with one of the electrodes 5421, 5422, and may also be configured to have an electrode arranged on the outer side of the protruding part 2121, like the electrodes 5422 in the measuring device 1D. Moreover, each electrode forming the back surface-side electrode 542 is not limited to the ring-shape and may be divided into a plurality of electrodes.

Also, the configuration of the measuring device 1E may include the casing 2A having the main body part 21A formed in a substantially circular shape as viewed from the back side, instead of the casing 2B having the main body part 21B formed in a substantially rectangular shape as viewed from the back side, and the foregoing configuration may be applied to the back surface 212 of the main body part 21A.

### Modifications of Embodiments

The invention is not limited to the embodiments. Modifications, improvements and the like within a range which can achieve the object of the invention are included in the invention.

In the first, second and third embodiments, the front surface-side electrode 541 has the two electrodes 5411, 5412, and the back surface-side electrode 542 has the two electrodes 5421, 5422. Also, in the second embodiment, the front surface-side electrode 541 has the four electrodes 541A to 541D, and the back surface-side electrode 542 has the two electrodes 5421, 5422. However, the invention is not limited to these. That is, it suffices that the first surface-side electrode arranged on the first surface of the main body part of the casing is formed by at least one electrode, that the second surface-side electrode arranged on the second surface as another surface is formed by at least one electrode, and that at least one of the first surface-side electrode and the second surface-side electrode has a plurality of electrodes. For example, the front surface-side electrode as the first surface-side electrode may have one electrode, and the back surface-side electrode as the second surface-side electrode may have two electrodes. Also, the back surface-side electrode may have three or more electrodes.

Also, the surface where an electrode is arranged is not limited to the front surface 211 and the back surface 212 and maybe another surface as well. For example, an electrode may be arranged on at least one of the right lateral surface 213 and the left lateral surface 214, instead of or in addition to the electrodes 5411, 5412 arranged on the front surface 211. In this case, if the fingers of the right hand RH are placed on the right lateral surface 213 and the left lateral surface 214 in such a way as to hold the main body part in the state where the measuring device is worn on the left wrist LW, the electrical continuity between the electrodes arranged on these lateral surfaces 213, 214 and the human body can be achieved. Similarly, an electrode may be arranged on at least one of the surfaces on the sides of the straps 28, 29, of the main body part.

Moreover, the electrodes which come into contact with the human body when the measuring device is worn is not limited to being provided in the main body part and may be in provided at least one of the straps 28, 29 forming the casings 2A, 2B. In this case, the electrodes and the main body part can be connected together by providing a signal line (electrical wire) along the straps 28, 29 on the inner side or the other side of the straps 28, 29.

In the respective embodiments, the electrode setting unit 97 sets working electrodes and a reference electrode used for the definitive measurement of an electrocardiogram, based on a measured impedance value and the result of the provisional measurement of an electrocardiogram using provisional working electrodes and a provisional reference electrode which are set. However, the invention is not limited to this. For example, the electrode used as a reference electrode may be decided in advance. Also, working electrodes and a reference electrode maybe selected and set, based on the result of the provisional measurement of an electrocardiogram without measuring a bioelectrical impedance value, or based on other conditions. If a reference electrode is selected and set from among the electrodes that are not used as working electrodes, there is no need to provide a reference electrode in advance and this reference electrode can be selected and set from among electrodes with low impedance values. Therefore, the detection accuracy of the electrocardiogram can be improved further.

Moreover, the result of the provisional measurement of an electrocardiogram carried out before setting working electrodes and a reference electrode used for the definitive measurement of an electrocardiogram may be obtained with one combination of provisional working electrodes and a provisional reference electrode. In other words, working electrodes and a reference electrode used for the definitive measurement may be set, based on an electrocardiogram of the user detected with one combination of provisional working electrodes and a provisional reference electrode. That is, there is no need to carry out the provisional measurement with all combinations of provisional working electrodes and a provisional reference electrode. Working electrodes and a reference electrode may be set, based on the result of the provisional measurement with at least one combination.

In the respective embodiments, the electrode setting unit 97 selects and sets working electrodes and a reference electrode, based on the waveform or the like of an electrocardiogram provisionally measured with each combination of provisional working electrodes and a provisional reference electrode. However, the invention is not limited to this. That is, the setting of working electrodes and a reference electrode may be carried out based on other conditions and methods. For example, these working electrodes and reference electrode may be set, based on the signal intensity of a detected electrocardiographic signal. Also, working electrodes may be set based on the temperature of an electrode when the human body touches the electrode.

In the respective embodiments, the electrodes 5411, 5412, 541A to 541D of the front surface-side electrode 541 are arranged in the electrode arrangement part 23 surrounding the display unit 61 arranged on the front surface 211. However, the invention is not limited to this. That is, these electrodes 5411, 5412, 541A to 541D need not necessarily be arranged at positions surrounding the display unit 61 and may be arranged in the state of being insulated from each other.

In the first embodiment, the electrodes 5411, 5412 of the front surface-side electrode 541 are separated from each other at the dividing positions DP1, DP2 situated on the straight line L1 (dividing positions DP1, DP2 at the positions of half past four and half past ten), and arranged at the lower left and upper right positions on the front surface 211. Meanwhile, in the second embodiment, the electrodes 541A to 541D of the front surface-side electrode 541 are separated from each other at the dividing positions DPA to DPD situated on the straight lines L1, L2 (dividing positions DPA to DPD at the positions of half past one, half past four, half past seven, and half past ten), and arranged at the upper, right-hand, lower, and left-hand positions on the front surface 211. However, the invention is not limited to these. That is, the arrangement of the electrodes forming the front surface-side electrode 541 can be changed when appropriate. For example, the respective electrodes maybe arranged in parallel along one direction (for example, the Y-direction or the X-direction) or maybe arranged at a part on the outer side of the display unit 61, along the circumferential direction of the display unit 61.

Also, the dividing positions DP1, DP2, DPA to DPD separating the electrodes 5411, 5412, 541A to 541D from each other are not limited to the foregoing positions and may be at other positions. For example, the dividing positions DP1, DPB may be situated between the direction of 4 o'clock and the direction of 5 o'clock about the center C1 of the front surface 211, and the dividing positions DP2, DPD may be situated between the direction of 10 o'clock and the direction of 11 o'clock about the center C1.

In addition, the respective electrodes need not necessarily be the same size. As in the case of the measuring device 1B, the shape of the respective electrodes is not limited to an arc-shape and may be other shapes.

In the first to third embodiments, the electrodes 5421, 5422 of the back surface-side electrode 542, too, are arranged concentrically about the center C2. However, the invention is not limited to this. That is, the arrangement of the respective electrodes 5421, 5422 may be changed when appropriate. For example, these electrodes may be arranged similarly to the electrodes 5411, 5412 or similarly to the electrodes 541A to 541D, on the back surface 212. The same applies to the back surface-side electrode 542 described in the fourth embodiment.

In the respective embodiments, the biological information detection unit 52 has the pulse wave detection unit 53, which detects a pulse wave of the user, in addition to the electrocardiographic measuring unit 54 and the impedance measuring unit 55. However, the invention is not limited to this. That is, the pulse wave detection unit 53 maybe omitted and the biological information detection unit 52 may be configured to further include a detection unit which detects other items of biological information (for example, blood pressure, blood sugar level, body temperature, and amount of perspiration). Also, the body motion information detection unit 51 may be omitted.

In the respective embodiments, the electrodes 5411, 5412, 541A to 541D forming the front surface-side electrode 541 are used only for the measurement of an electrocardiogram of the user. However, this is not limiting. For example, the electrodes may be configured to be usable as buttons forming the operation unit 4 except the time of measuring an electrocardiogram.

In the respective embodiments, the main body parts 21A, 21B forming the casings 2A, 2B are worn on the human body with the pair of straps 28, 29 as a wearing member. However, the invention is not limited to this. That is, any configuration can be employed for the wearing member, provided that the measuring devices 1A to 1C can be worn on a human body. Also, as described above, the straps 28, 29 may be integrated with the main body parts 21A, 21B.

In the respective embodiments, the measuring devices 1A to 1E are wearable devices which are configured in the shape of a wristwatch and can be worn on the left wrist LW of the user. However, this is not limiting. The shape of the measuring devices may be other shapes such as substantially rectangular parallelepiped. In this case, the straps 28, 29 maybe omitted. Also, the wearing site of the measuring devices 1A to 1E is not limited to the left wrist LW and may be other positions such as the right wrist or an ankle.

### REFERENCE SIGNS LIST

1A, 1B, 1C, 1D, 1E . . . biological information measuring device, 2A, 2B . . . casing, 21A, 21B . . . main body part, 211 . . . front surface (first surface), 212 . . . back surface (second surface), 23 . . . electrode arrangement part, 28, 29 . . . strap (wearing member), 52 . . . biological information detection unit, 53 . . . pulse wave detection unit, 541 . . . front surface-side electrode (first surface-side electrode), 5411, 5412, 541A, 541B, 541C, 541D . . . electrode, 542 . . . back surface-side electrode (second surface-side electrode), 5421, 5422 . . . electrode, 547 . . . electrocardiographic detection unit, 61 . . . display unit, 9 . . . control unit.

1. A biological information measuring device comprising:
a biological information detection unit which detects biological information of a user;
a control unit which controls the biological information detection unit; and
a casing which houses the biological information detection unit and the control unit,
wherein the biological information detection unit includes
a first surface-side electrode arranged on a first surface of the casing,
a second surface-side electrode arranged on a second surface which is a different surface from the first surface of the casing, and
an electrocardiographic detection unit which detects an electrocardiogram of the user, using the first surface-side electrode and the second surface-side electrode,
at least one of the first surface-side electrode and the second surface-side electrode has a plurality of electrodes, and
the control unit sets one electrode of the first surface-side electrode and one electrode of the second surface-side electrode as working electrodes, sets, as a reference electrode, one of the electrodes that are not set as the working electrodes, and causes the electrocardiographic detection unit to measure an electrocardiogram of the user, based on a current detected with the working electrodes.

2. The biological information measuring device according to claim 1, wherein
the control unit sets the reference electrode, based on an impedance value based on a voltage value of a current which is outputted to the first surface-side electrode and electrically continues to the second surface-side electrode.

3. The biological information measuring device according to claim 1, wherein
the control unit sets the working electrodes and the reference electrode used for measurement of an electrocardiogram of the user, based on an electrocardiogram of the user detected with at least one combination of electrodes in which one electrode of the first surface-side electrode and one electrode of the second surface-side electrode are set as provisional working electrodes and in which one of the electrodes that are not set as the provisional working electrodes is set as a provisional reference electrode.

4. The biological information measuring device according to claim 1, wherein
the casing includes
a main body part, and
a wearing member which allows the main body part to be worn at a wearing site of the user,
the second surface is a surface in contact with the wearing site when the main body part is worn at the wearing site with the wearing member, and
the first surface is a surface opposite to the second surface.

5. The biological information measuring device according to claim 4, further comprising
a display unit arranged on the first surface of the main body part,
wherein the main body part has an electrode arrangement part which is arranged on the first surface and surrounds the display unit, and
the first surface-side electrode is arranged in the electrode arrangement part.

6. The biological information measuring device according to claim 5, wherein
the first surface-side electrode includes a plurality of electrodes, and
the plurality of electrodes is separated from each other at a position between a direction of 4 o'clock and a direction of 5 o'clock and a position between a direction of 10 o'clock and a direction of 11 o'clock, as viewed from a position directly opposite the first surface.

7. The biological information measuring device according to claim 1, wherein
the biological information detection unit has a pulse wave detection unit which detects a pulse wave of the user.
